(19) United States
(12) Patent Application Publication
(10) Pub. No.: US 2022/0226830 A1
(43) Pub. Date: Jul. 21, 2022

(54) POINT OF CARE DROPLET DIGITAL PCR

(71) Applicant: University of Notre Dame du Lac, South Bend, IN (US)

(72) Inventors: Hsueh-Chia Chang, South Bend, IN (US); Liao Chen, South Bend, IN (US); Chenguang Zhang, South Bend, IN (US); FNU Vivek, South Bend, IN (US)

(21) Appl. No.: 17/577,550

(22) Filed: Jan. 18, 2022

Related U.S. Application Data
(60) Provisional application No. 63/138,857, filed on Jan. 19, 2021.

(51) Int. Cl. B01L 9/00; C12Q 1/686

(52) U.S. Cl. CPC B01L 9/543; C12Q 1/686

(57) ABSTRACT

Described herein are methods and compositions for point of care droplet digital PCR (ddPCR) using large uniform droplets that can be visually analyzed for detection. In one embodiment, the ddPCR reaction is prepared by dispensing a plurality of large uniform droplets using a modified pipette tip having a specific aspect ratio and one or more surfactants in the aqueous phase.

### CROSS REFERENCE TO RELATED APPLICATIONS

This application claims priority to U.S. Provisional Patent Application No. 63/138,857, filed on Jan. 19, 2021, the contents of which are incorporated by reference herein in its entirety.

### FEDERALLY SPONSORED RESEARCH

This invention was made with government support under grant number CA241684 awarded by the National Institutes of Health. The government has certain rights in the invention.

### TECHNICAL FIELD

Described herein are methods and compositions for point of care droplet digital PCR (ddPCR) using large uniform droplets that can be visually analyzed for detection. In one embodiment, the ddPCR reaction is prepared by dispensing a plurality of large uniform droplets using a modified pipette tip having a specific aspect ratio and one or more surfactants in the aqueous phase.

### BACKGROUND

Reverse transcription polymerase chain reaction (RT-PCR) is the standard for SARS-CoV-2 virus detection from nasopharyngeal swabs and/or saliva samples. However, the need to conduct this assay in laboratories and the resulting long transport time have become a bottleneck on the control of rapidly spreading pandemic with high positivity rates. Even with its expensive and lab-bound optical instruments, the false negative rate of RT-PCR detection especially for the newly infected samples with low viral concentration is not satisfactory. It is known that compared with RT-PCR, droplet digital polymerase chain reaction (ddPCR) has better sensitivity by partitioning the samples into large numbers of droplets to enrich the virus RNA and also mitigate the influences of PCR inhibitors. Their simpler optical detectors also suggest ddPCR products can be used at the Point-of-Care (POC), without transportation to laboratories.

A viable rapid POC ddPCR COVID screening test has not been reported. Alternatives to the bulky and expensive micropumps in commercial products have been proposed. However, the main challenge for POC ddPCR is the droplet size and the related lab-bound optical detection platform. Commercial ddPCR products use carefully designed flow focusing (for example, products by Bio-Rad) and step emulsification (by Stilla) microfluidic chips typically generate droplets that are picoliters to several nanoliters in volume. The current commercial RNA extraction kit yields about 1-100 copies of virus RNA from early-stage patients in a typical 10 μL extract for PCR tests. Such low viral load is near the limit-of-detection of many PCR tests. Once the extract is combined with the PCR mixture, a total of 20-30 μL will then need to be converted into droplets, corresponding to 10⁵ to 10⁷ droplets. The search for fewer than 100 positive droplets out of approximately one million droplet requires spreading a large monolayer of droplets if panned imaging is used or a flow format with individual droplet interrogation. Both are time-consuming, equipment-intensive, and error-prone, leading to sensitivity corruption even with the expensive and bulky optical detectors. They are not POC operations. It is possible for some droplet generation technologies to generate larger and fewer droplets. However, large (>100 microns) droplets have thermal stabilization issues during PCR amplification, leading to inaccurate quantification. There is also a detection issue with larger droplets. The fluorescence intensity of the PCR reporter is a function of the droplet size, as it is determined by the concentration of the reporter. Hence, for a typical 30 to 40-cycle assay, the activated number of reporters in a droplet must yield an overall concentration over one droplet that is higher than the threshold required by a particular detector. Ideally, with droplets that are larger than 100 microns, a high enough fluorescence intensity may permit quantification by eye with blue light illumination (transilluminator) and imaging by a smartphone camera. Visual quantification would completely remove the need for expensive and bulky optical detectors and enable a rapid POC ddPCR test, if the bulky/expensive micropump is also eliminated.

What is needed is a new rapid, high-throughput, and inexpensive approach to generate large uniform droplets suitable for ddPCR assays.

### SUMMARY

One embodiment described herein is a pipette tip for performing rapid droplet digital Polymerase Chain Reaction (ddPCR) assays, the pipette tip comprising a proximal end having a first orifice adapted to be operably connected to a pipette, an elongated main body having a cylindrical sidewall forming an interior axial bore and tapering longitudinally from the proximal end to a distal end having a second orifice, the second orifice being in communication with the interior axial bore and adapted to allow the passage of fluid therethrough, and the second orifice having an elliptical cross-section with an orifice aspect ratio greater than about 3.5. In one aspect, the second orifice has an orifice aspect ratio ranging from about 3.5 to about 6. In another aspect, the second orifice has an orifice aspect ratio ranging from about 3.5 to about 5. In another aspect, an interior sidewall of the interior axial bore is coated with one or more surfactants. In another aspect, the one or more surfactants comprises a fluorosurfactant in an oil phase. In another aspect, the one or more surfactants comprises a water-soluble surfactant in an aqueous phase.

Another embodiment described herein is a method for modifying a commercially available pipette tip, the method comprising: selecting a commercially available pipette tip; and deforming the pipette tip orifice circumference to have an orifice aspect ratio of equal to or greater than about 3.5. Another embodiment described herein is a modified pipette tip produced by the preceeding method.

Another embodiment described herein is a method for manufacturing a pipette tip having an orifice aspect ratio of equal to or greater than about 3.5, the method comprising: providing an injection mold comprising a mold cavity having a geometry configured to generate a pipette tip having an orifice aspect ratio of equal to or greater than about 3.5; contacting the mold cavity with a molten polymer; incubating the molten polymer in the mold cavity for sufficient time to allow the molten polymer to cool and solidify in the mold cavity; and releasing the formed pipette tip from the mold cavity after cooling. Another embodiment described herein is a modified pipette tip produced by the preceeding method.

Another embodiment described herein is a method of rapidly generating a plurality of large uniform droplets for performing droplet digital Polymerase Chain Reaction (ddPCR) assays, the method comprising: providing a pipette affixed to a pipette tip having a tip orifice aspect ratio of equal to or greater than about 3.5 and a surfactant coating on an interior axial bore surface of the pipette tip; loading the pipette tip with a PCR reaction mixture; and continuously expelling the PCR reaction mixture from the pipette tip, thereby generating a plurality of large uniform droplets. In one aspect, the pipette tip having an orifice aspect ratio of equal to or greater than about 3.5 is generated by: providing a commercially available pipette tip; and applying sufficient pressure between two surfaces to a longitudinal section of the pipette tip adjacent to and comprising the tip orifice for sufficient time to deform the orifice circumference to have an orifice aspect ratio of equal to or greater than about 3.5. In another aspect, the surfactant coating on the interior axial bore of the pipette tip is prepared by contacting the interior axial bore surface of the pipette tip with one or more surfactants, incubating the surfactant for a sufficient period of time to adhere to the interior axial bore surface; and expelling the surfactant.

Another embodiment described herein is a method of rapidly generating a plurality of large uniform droplets for performing droplet digital Polymerase Chain Reaction (ddPCR) assays, the method comprising: providing a commercially available pipette tip; deforming the pipette tip orifice circumference to have an orifice aspect ratio of equal to or greater than about 3.5; contacting an interior sidewall of an interior axial bore of the pipette tip with one or more surfactants to generate a surfactant coating; loading the pipette tip having an orifice aspect ratio of equal to or greater than about 3.5 and a surfactant coating with a PCR reaction mixture; and continuously expelling the PCR reaction mixture from the pipette tip, thereby generating a plurality of large uniform droplets. In one aspect, deforming the pipette tip orifice circumference comprises applying sufficient pressure between two surfaces to a longitudinal section of the pipette tip adjacent to and comprising the tip orifice for sufficient time to deform the orifice circumference to have an orifice aspect ratio of equal to or greater than about 3.5. In another aspect, the one or more surfactants comprises a fluorosurfactant in an oil phase. In another aspect, the fluorosurfactant comprises RAN-008, Krytox, or a combination thereof, in the oil phase of HFE-7500 oil. In another aspect, the fluorosurfactant is present in the oil phase at a concentration ranging from about 1 w/v % to about 5 w/v %. In another aspect, the method further comprises a water-soluble surfactant in an aqueous phase to stabilize the droplets in the oil phase during the ddPCR assay. In another aspect, the water-soluble surfactant is polyoxyalkylene block copolymer surfactant Pluronic F127, Poloxamer 188, or a combination thereof. In another aspect, the polyoxyalkylene block copolymer surfactant is present in the aqueous phase at a concentration of 0.01 w/v %. In another aspect, the polyoxyalkylene block copolymer surfactant is present in the aqueous phase at a concentration ranging from about 0.01 w/v % to about 1.5 w/v %. In another aspect, the plurality of large uniform droplets comprises individual droplets having a size ranging from about 100 μm to about 400 μm. In another aspect, the plurality of large uniform droplets comprises individual droplets having a size ranging from about 150 μm to about 350 μm. In another aspect, the plurality of large uniform droplets comprises individual droplets having a size ranging from about 150 μm to about 200 μm. In another aspect, the plurality of large uniform droplets comprises between about 1,000 to about 10,000 monodispersed droplets. In another aspect, the plurality of large uniform droplets comprises about 5,000 monodispersed droplets. In another aspect, the plurality of large uniform droplets is generated in less than 5 min. In another aspect, the plurality of large uniform droplets has a coefficient of variation (CV) of less than 3%. In another aspect, the plurality of large uniform droplets has a CV of about 2.3%. In another aspect, the plurality of droplets is visually analyzed following the ddPCR assay using a camera or microscope. In another aspect, the pipette tip has an orifice aspect ratio ranging from about 3.5 to about 6. In another aspect, the pipette tip has an orifice aspect ratio ranging from about 3.5 to about 5. In another aspect, the method accelerates the total ddPCR assay processing time to less than about 1.5 hr. In another aspect, the method has a limit of detection (LoD) of 3.8 copies per 20 μL reaction. In another aspect, the method has a dynamic range of 4 to 100 copies. In another aspect, the method is performed outside of a centralized lab and is suitable for point-of-care (POC) quantification applications.

Another embodiment described herein is a method of detecting or measuring the presence of a virus in a subject by rapidly generating a plurality of large uniform droplets for performing rapid droplet digital Polymerase Chain Reaction (ddPCR) assays, the method comprising: obtaining a sample from the subject; processing the sample for ddPCR analysis; loading a pipette tip having an orifice aspect ratio of equal to or greater than about 3.5 and a surfactant coating with a PCR reaction mixture containing the processed sample; continuously expelling the PCR reaction mixture from the pipette tip, thereby generating a plurality of large uniform droplets; performing the ddPCR assay; and visually analyzing the plurality of droplets following the ddPCR assay to detect or measure the presence of a virus in the sample. In one aspect, the sample comprises whole blood, serum, plasma, urine, tears, sweat, saliva, nasopharyngeal fluid, lymph, cerebrospinal fluid, fecal extract, cellular or tissue extracts, or any other aqueous sample. In another aspect, the virus is SARS-CoV-2. In another aspect, the plurality of large uniform droplets is visually analyzed using a camera or microscope.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, Panels (1)-(4) show a scheme of the point of care (POC) droplet digital PCR methodology. FIG. 1, Panel (1) shows the fabrication of head-flattened pipette tips, FIG. 1, Panel (2) shows the preparation of uniform large microdroplets using manual pipetting with flattened pipette tips. FIG. 1, Panel (3) shows that large microdroplets are stabilized for PCR thermal cycling. FIG. 1, Panel (4) shows the analysis and detection of positive reactions in the large microdroplets by visual inspection or using a smartphone with a transilluminator.

FIG. 2 shows exemplary aspect ratios of 1, 2, 3.5, 5, 6, 7, 8, and 10, of a 1-inch circle; an ellipse with a major axis of 1 and minor axis of 0.5; an ellipse with a major axis of 1 and minor axis of 0.285; an ellipse with a major axis of 1 and minor axis of 0.2, an ellipse with a major axis of 1 and minor axis of 0.166, an ellipse with a major axis of 1 and minor axis of 0.143, an ellipse with a major axis of 1 and minor axis of 0.125, an ellipse with a major axis of 1 and minor axis of 0.1, respectively. All axis values are inches for this example.

FIG. 3A-F show droplet generation with pipette tips. FIG. 3A shows the top-view (top) and cross-section view (bottom) of a commercial micro-loading pipette tip. FIG. 2B shows the generated water droplets and FIG. 3C shows the ddPCR reaction mixture droplets. FIG. 3D shows the top-view (top) and cross-section view (bottom) of the pipette tip with a flattened head. FIG. 3E shows the generated water droplets and FIG. 3F shows the ddPCR reaction mixture droplets.

FIG. 4A-D show water droplets generated using pipette tips having various orifice aspect ratios. FIG. 4A shows a pipette tip with an aspect ratio of ˜1.1 (top) and the generated water droplets (bottom) having a diameter of 685±147 μm. FIG. 4B shows a pipette tip with an aspect ratio of ˜2 (top) and the generated water droplets (bottom) having a diameter of 687±151 μm. FIG. 4C shows a pipette tip with an aspect ratio of ˜3.5 (top) and the generated water droplets (bottom) having a diameter of 347±23 μm. FIG. 4D shows a pipette tip with an aspect ratio of ˜4 (top) and the generated water droplets (bottom) having a diameter of 178±6 μm. Uniform droplets are easily pipetted out when the aspect ratio is ˜3.5 or greater. Oil phase: HFE-7500 (3M Novec); surfactant in the oil phase: 2 wt % RAN-008 surfactant.

FIG. 5 shows a graph of mean droplet radius versus the aspect ratio of the pipette tip orifice.

FIG. 6A-F show pipette droplet generation in HFE-7500 oil with 2 v/v % Krytox 157 FSH as surfactant and increasing concentrations of polyoxyalkylene block copolymer surfactant Pluronic® F127 or Poloxamer 188 (P188). FIG. 6A shows that pure water droplets are not stable and coalesce immediately after generation. FIG. 6B-D show that by adding increasing concentration of Pluronic® F127 surfactant to 0.001, 0.01, and 1 w/v %, respectively, the droplets become more uniform and stable. FIG. 6E shows aqueous droplets in HFE-7500 oil with 2 v/v % Krytox 157 FSH as surfactant, and 0 wt % P188 (left) or 0.5 wt % P188 (right). Polyoxyalkylene block copolymer P188 helps stabilize water droplets similar to F127. FIG. 6F shows successful droplet digital PCR conducted in droplets stabilized with 0.5 w/v % P188.

FIG. 7A-E show efficient ddPCR with stabilized large microdroplets. FIG. 7A shows that pristine droplets in oil are not stable and coalesce severely after 40 PCR cycles (right). FIG. 7B shows that the larger microdroplets are stabilized with the addition of block copolymer Pluronic® F127 (≥0.01 w/v %) and do not coalesce after 40 PCR cycles (right). FIG. 7C-D show stabilized droplets after 40 PCR cycles as imaged by a table-top fluorescence microscope. FIG. 7E shows identical droplets imaged by a handheld fluorescence mini-microscope (left) and a smartphone camera with a transilluminator (right). The stabilized large droplets are useful for ddPCR and are large enough to enable visual analysis or analysis using a smartphone camera to determine positive reactions.

FIG. 8A-E show that the point-of-care (POC) ddPCR methodology works for low target copy samples. FIG. 8A shows a handheld fluorescent microscope image (left) and a smartphone camera image (right) of a sample with nominally 2 template copies in 20 μL PCR reactions, with the arrows pointing to positive droplets. FIG. 8B shows a handheld fluorescent microscope image (left) and a smartphone camera image (right) of a sample with nominally 10 template copies in 20 μL PCR reactions. FIG. 8C shows a handheld fluorescent microscope image (left) and a smartphone camera image (right) of a sample with nominally 25 template copies in 20 μL PCR reactions. FIG. 8D shows a handheld fluorescent microscope image (left) and a smartphone camera image (right) of a sample with nominally 50 template copies in 20 μL PCR reactions. FIG. 8E shows a handheld fluorescent microscope image (left) and a smartphone camera image (right) of a sample with nominally 100 template copies in 20 μL PCR reactions. The large microdroplets can be imaged using typical smartphone cameras and the results are consistent with those imaged using a handheld fluorescent mini-microscope. The scale bars are 4 mm.

FIG. 9 shows the detection of N target region.

FIG. 10 shows the detection of ORF1ab target region.

FIG. 11A-E show fluorescence intensity comparison between bulk PCR and droplet PCR reactions. FIG. 11A shows droplets generated after 40 cycles of bulk PCR with 10 target copies (top) and 0 target copy (bottom). FIG. 11B shows droplets from 40 cycles of droplet PCR with 10 target copies. FIG. 11C shows droplets generated after 30 cycles of bulk PCR with 10 target copies (top) and 0 target copy (bottom). FIG. 11D shows droplets from 30 cycles of droplet PCR with 10 target copies. FIG. 11E shows a bar graph of normalized fluorescence intensity comparing bulk and droplet PCR. The scale bars are 500 μm.

FIG. 12 shows smartphone camera images of droplet samples with 100 target copies after 40 PCR cycles (left) and 30 PCR cycles (right). After 30 or more PCR cycles, positive droplets become discernable.

FIG. 13A-D show smartphone camera images of POC ddPCR. FIG. 13A shows an image of a saliva-free sample with nominally 10 target copies. FIG. 13B shows an image of a 10 v/v % saliva-doped sample with no target copies. FIG. 13C shows an image of a 10 v/v % saliva-doped sample with nominally 10 target copies. FIG. 13D shows an image of a 20 v/v % saliva-doped sample with nominally 10 target copies showing polydispersed droplets and no positive droplets. The tolerance of saliva impurities indicates that POC ddPCR is possible for RNA-extraction-free tests. The scale bars are 4 mm.

FIG. 14A-C show a droplet generated by a commercial gel-loading flat pipette tip for droplet digital PCR. FIG. 14A shows the cross-section of the commercial gel-loading flat pipette tip orifice. Following 40 cycles of PCR, droplets are imaged by a table-top fluorescence microscope (FIG. 14B). FIG. 14C shows that droplets are also imaged by a handheld mini-microscope (left) and a smartphone camera with a transilluminator (right). The commercially available flat pipette tips seem to provide a promising approach for droplet generation and subsequent droplet digital PCR.

FIG. 15A-B show droplets generated from either periodical (FIG. 15A) or continuous (FIG. 15B) pipetting. FIG. 15A shows droplets generated by periodical pipetting with a flow rate of 2 μL/s for 1 second and no flow for 2 seconds in each period. FIG. 15B shows droplets generated by continuous pipetting with a flow rate of 2 μL/s.

FIG. 16A-B show images of parallel droplet generation via a multi-channel pipette gun. FIG. 16A shows an eight-channel pipette gun generating droplets inside eight PCR 200-μL tubes in parallel. FIG. 16B shows a sample of the generated droplets.

FIG. 17A-B show images of a transparent droplet reservoir for point-of-care digital assays that was produced by 3D printing. FIG. 17A shows the 3D model of the droplet reservoir and the two corresponding cross-sections. The droplet reservoir has two outlets which can be blocked by two matched plugs after droplet generation/loading. Inside the droplet reservoir, the height of the interior chamber decreases from periphery to the center so that droplets are pinned and immobilized away from the walls for easy handling and imaging. FIG. 17B shows a 3D-printed droplet reservoir with matched plugs (left) and droplets immobilized inside the droplet reservoir for imaging (right). The droplets stay still and immobilized away from the walls inside the droplet reservoir because of the changing interior chamber height. The exterior dimensions of the droplet reservoir are 20 m×20 m×2.5 mm.

FIG. 18 shows images of pipette droplet generation for rapid isothermal (20 min at ˜40° C.) recombinase polymerase amplification (RPA) comparing droplets with a blank control sample (left) and droplets with a positive sample (right).

FIG. 19A-C show a method for tip modification to prepare head-flattened pipette tips using torque via an easily assembled tool. FIG. 19A shows two separate images of the simple tool that consists of a 3D-printed case, a 3D-printed plate, a steel clamp, and a torque screwdriver (left). The purpose of the tool is to standardize the manual deforming of a commercial extended-length or gel-loading pipette tip having a round orifice to a tip having a flattened orifice with a specific cross-section aspect ratio. When using the tool, a pipette tip is inserted between the plate (or a glass slide) and the substrate (right). The plate (or glass slide) cannot rotate freely once assembled with the 3D-printed case and will transfer the torque to pressure to deform the orifice of the pipette tip. FIG. 19B shows a graph of the cross-section aspect ratio of a head-flattened pipette tip versus applied torque using the tool shown in FIG. 19A. The graph represents a type of calibration curve for a modified pipette tip orifice aspect ratio in terms of applied torque. Based on the calibration curve, specific aspect ratios could be roughly achieved by applying the corresponding torques. FIG. 19C shows droplets generated by flattened pipette tips prepared under 15 torque using the tool shown in FIG. 19A. DI water droplets (left), and 0.5 w/v % P188 aqueous solution droplets (right) were both in HFE-7500 oil with 2 wt % RAN-008-fluorosurfactant.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive. As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

“Sample” or “test sample” as used herein can mean any sample in which the presence and/or level of a target is to be detected, determined, or measured as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasopharyngeal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or subject or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the term “biomarker” or “target” refers to a measurable indicator of some biological state or condition, or to a substance, the presence of which indicates the existence of a living organism. Biomarkers can be measured and evaluated to examine normal biological processes, pathogenic processes, or pharmacologic responses to a therapeutic intervention. As used herein, the term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

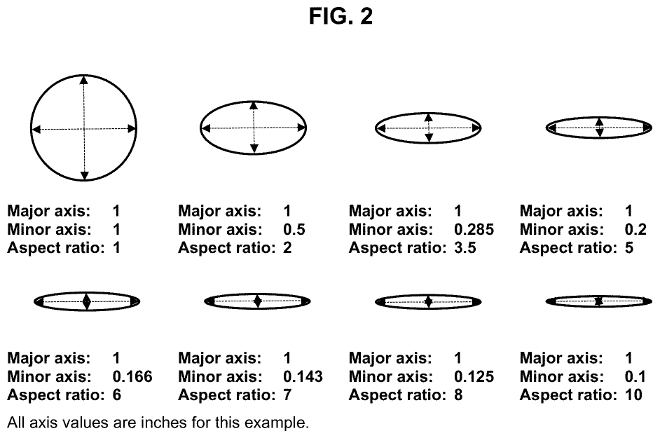

As used herein the term “aspect ratio” or “cross-section aspect ratio” refers to the dimensional ratio of width to height (w:h). In one aspect, “aspect ratio” refers to the ratio of the major axis to the minor axis of a circular or elliptical opening, for example, the orifice of a pipette tip. For example, an orifice with an aspect ratio of 1:1 is a circle. An elliptical orifice with a major axis of 1 and a minor axis of 0.5 has an aspect ratio of 2. An elliptical orifice with a major axis of 1 and a minor axis of 0.285 has an aspect ratio of 3.5. An elliptical orifice with a major axis of 1 and a minor axis of 0.2 has an aspect ratio of 5. An elliptical orifice with a major axis of 1 and a minor axis of 0.1 has an aspect ratio of 10. See FIG. 2. Axis values can be any unit of measurement (e.g., inches, cm, mm, or μm). In one aspect, the aspect ratio is 1, 2, 3, 4, 5, 6, 7, 8, 9, or 10. In another aspect, the aspect ratio is about 2-10, 2-8, 2-6, 2-5, 2-4, 3-10, 3-8, 3-6, 3-5, 3-4, 4-10, 4-8, 4-6, 4-5, 5-10, 5-8, or 5-6, including all integers within the ranges. In another aspect, the aspect ratio is about 2.0, 2.1, 2.2, 2.3, 2.4, 2.5, 2.6 2.7, 2.8, 2.9, 3.0, 3.1, 3.2, 3.3, 3.4, 3.5, 3.6 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, or even greater. In one aspect, the aspect ratio is about 3.5. In another aspect, the aspect ratio is greater than or equal to about 3.5 (≥˜3.5). In another aspect, the aspect ratio is about 3.0-5.0, 3.1-5.0, 3.2-5.0, 3.3-5.0, 3.5-5.0, 3.6-5.0, 3.7-5.0, 3.8-5.0, 3.9-5.0, 3.0-6.0, 3.1-6.0, 3.2-6.0, 3.3-6.0, 3.5-6.0, 3.6-6.0, 3.7-6.0, 3.8-6.0, 3.9-6.0, 4.0-6.0, 4.0-5.0, 4.1-5.0, 4.2-5.0, 4.3-5.0, 4.5-5.0, 4.6-5.0, 4.7-5.0, 4.8-5.0, 4.9-5.0, 5.0-6.0, 5.1-6.0, 5.2-6.0, 5.3-6.0, 5.4-6.0, 5.5-6.0., 5.6-6.0, 5.7-6.0. 5.8-6.0, or 5.9-6.0. In one aspect, the aspect ratio is about 3.5 to about 6. In one aspect, the aspect ratio is about 3.5 to about 5. In one aspect, the aspect ratio is about 3.5 to about 4.

As used herein, the term “monodispersed” refers to two or more liquid droplets having substantially the same size, substantially the same diameter, substantially the same radius, or substantially a uniform size in dispersed phase. Monodispersed liquid droplets can vary in size by, for example, ±0.1 μm, ±0.5 μm, ±1 μm, ±2 μm, ±5 μm, ±10 μm, ±20 μm, ±50 μm, ±60 μm, ±70 μm, ±80 μm, ±90 μm, ±100 μm, ±200 μm, ±300 μm, ±400 μm, ±500 μm, ±600 μm, ±700 μm, ±800 μm, ±900 μm, or ±1000 μm, or any integer within the range of 0.1-1000 μm. When a range of monodispersed liquid droplets is recited, the range refers to the size of all droplets, and not the different sizes of droplets. For example, “monodispersed liquid droplets having a diameter ranging from about 0.1 μm to about 1000 μm” means, for a particular diameter of liquid droplet within the range cited, all, a preponderance, or an average of the dispersed droplets have the same diameter, or a substantially similar diameter, for each of the droplet diameters in the range recited above.

As used herein, the term “PCR reagent” or “PCR reaction mixture” is a term given ordinary meaning to a person skilled in the art and can include one or more PCR reagents. For example, a PCR reagent or PCR reaction mixture can comprise a polymerase enzyme (typically Taq Polymerase), template DNA, primers, deoxynucleotide (dNTP), MgCl₂, cofactors, and physiological buffers. For reverse-transcription PCR (rt-PCR), the PCR reagents or PCR reaction mixture can also comprise a reverse-transcription polymerase.

As used herein, the term “surfactant” refers to a surface-active agent capable of reducing the surface tension of a liquid in which it is dissolved, and/or the interfacial tension with another phase. A surfactant may incorporate both a hydrophilic portion and a hydrophobic portion, which may collectively confer a dual hydrophilic-hydrophobic character on the surfactant. In some embodiments, fluorosurfactants, or fluorinated surfactants, are used. Fluorosurfactants are organofluorine chemical compounds that have multiple fluorine atoms. They can be either polyfluorinated or perfluorinated. In some cases, the fluorosurfactant can be non-ionic. In some cases, the fluorosurfactant is a fluorosurfactant to the oil phase of the pristine oil HFE-7500 Engineered Fluid (3M Novec), having the formula C₉H₅F₁₅O. In one aspect of the invention, the fluorosurfactant is RAN-008-FluoroSurfactant (RAN Biotechnologies), having the formula PFPE-PEG/PEO-PFPE^{[1}]. In another aspect, the fluorosurfactant is Krytox® 157 FSH (DuPont), having the formula PFPE-COOH^{[2}]. In other embodiments, a water-soluble surfactant is used to stabilize droplets in oil during the ddPCR assay. In one aspect, the water-soluble surfactant is the polyoxyalkylene block copolymer surfactant Pluronic® F127 (Sigma-Aldrich), having the formula PPO-PEO-PPO[³]. In another aspect, the water-soluble surfactant is polyoxyalkylene block copolymer surfactant Poloxamer 188 (P188). Poloxamer 188 is a polyoxyalkylene block copolymer that has a slightly different molecular weight of the component blocks compared to Pluronic® F127. For droplet stabilization, Poloxamer 188 works just as good as Pluronic® F127. Other polyoxyalkylene block copolymers, including P123 and F108, can be used for droplet stabilization; however, Pluronic® F127 and Poloxamer 188 have been found to work the best. The superscript bracketed numbers refer to the surfactants shown in Table 1, below.

As used herein, the term “oil phase” refers to any liquid compound or mixture of liquid compounds that is immiscible with water. The oil used may be or include at least one of silicone oil, mineral oil, hydrocarbon oil, fluorocarbon oil, vegetable oil, or a combination thereof, among others. Any other suitable components may also be present in the oil phase, such as at least one surfactant, reagent, other additive, preservative, particles, or any combination thereof.

As used herein, the term “aqueous phase” refers to any liquid miscible with water. That is, aqueous phase can be any liquid that when mixed with water at room temperature, forms a stable single-phase solution. In some embodiments, the aqueous phase can comprise one or more physiologically acceptable reagents and/or solvents, etc. Some non-limiting examples of aqueous phase include water, DMF, DMSO, methanol, or ethanol.

Rapid Point-of-Care (POC) quantification of low virus RNA load would significantly reduce the turnaround time for the PCR test and help contain a fast-spreading epidemic. Herein, a droplet digital PCR (ddPCR) platform is demonstrated that can achieve this sensitivity and rapidity without bulky lab-bound equipment. The key technology is a flattened pipette tip with an elliptical cross-section, which extends a high aspect-ratio microfluidic chip design to pipette scale, for rapid (<5 min) generation of several thousand monodispersed droplets ˜150 to ˜350 μm in size with a coefficient of variation (CV) of ˜2.3%. A block copolymer surfactant (polyoxyalkylene Pluronic® F127 or Poloxamer 188) is used to further stabilize these large droplets in oil during thermal cycling. At this droplet size and number, positive droplets can be counted by eye or imaged by a smartphone with appropriate illumination/filtering to accurately quantify up to 100 target copies. Herein, a 2019 nCoV-PCR assay is demonstrated having a limit of detection (LoD) of 3.8 copies per 20 μL sample and a dynamic range of 4 to 100 copies. The ddPCR platform is shown to be inhibitor-resistant in a preliminary RNA-extraction-free saliva test. It represents a rapid 1.5-hour POC quantitative PCR test that requires just a pipette equipped with elliptical pipette tip, a commercial portable thermal cycler, a smartphone and a portable transilluminator—without bulky and expensive micropumps and optical detectors that prevent POC application.

A POC ddPCR virus test requires an optimum droplet size and an optimum number of droplets. The droplets must be small enough so that the reporter fluorescent intensity is visible to the eye or a smart phone camera, to eliminate the lab-bound optical detectors, and so that Poisson statistics still stipulates no more than one molecule per droplet such that the number of molecules can be estimated by counting the positive droplets. The droplets must be large enough so that the positive droplets can be easily discerned by eye in the entire droplet population within one smartphone imaging frame. These large droplets must be stable during thermal cycling and should be easily generated without an expensive lab-bound commercial micropump.

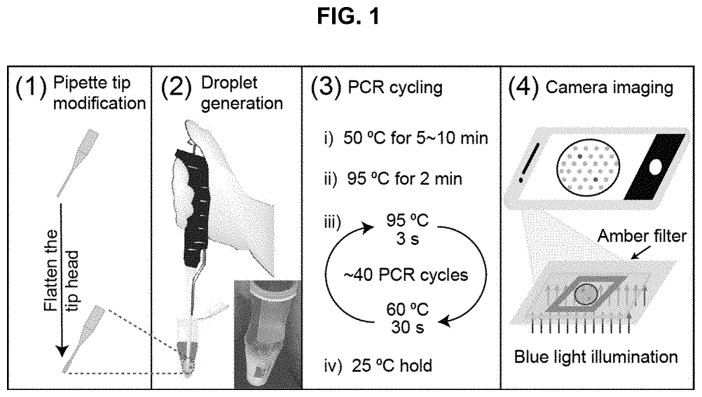

A low-cost POC droplet digital PCR platform is demonstrated herein (see FIG. 1). Approximately ˜1000 to ˜10,000 large microdroplets of optimal size (˜150 to 350 μm) are generated by head flattened pipette tips in about 5 minutes. The head-flattened pipette tip has an elliptic cross-section and is prewetted by oil. It is a pipette-tip/capillary version of an earlier design for a high aspect ratio droplet-generating microfluidic chip. The wetting oil film produce two long oil/water menisci at the sides of the pipette tip with a high curvature that is the same as the pipette tip wall. The high capillary pressure produced by these two side menisci control droplet pinching and produce monodispersed droplets. In contrast to the longitudinal curvature (capillary pressure) that stabilizes pinching of the extruding jet into droplets, the side curvature (capillary pressure) actually drives the pinching. An axisymmetric interface hence pinches off with equal contribution from these two opposing capillary pressures and produces non-monodispersed droplets with satellite droplets due to the competition. An interface with an elliptical cross section, however, favors the side capillary pressure that drives pinch off and hence produces monodispersed droplets, when the aspect ratio exceeds a critical value of 3.5.

The static capillary mechanism also ensures that the droplet generation is insensitive to the pipetting flow rate and the droplet diameter is proportional to the width of the pipette tip orifice. Precise control of the flow rate is hence unnecessary, as in flow focusing and step emulsification. As a result, monodispersed large microdroplets are easily prepared by pipette guns equipped with head-flattened tips into standard 200 μL PCR tubes loaded with fluorocarbon oil. The sample utilization is ˜100% and the generated droplets in PCR tubes are immediately ready for thermal cycling. These large droplets are not stable during PCR cycling and will coalesce. The thermal stability issue of large droplets is typically solved with very high concentration of surfactants, specially designed surfactants or crosslinking into gel beads. Inspired by the versatile micelle formation and aggregation/patterning phenomena of the biocompatible polyoxyalkylene block copolymer F127 solution with respect to temperature changes, the F127 solution was introduced into the aqueous phase (≥0.01 w/v %) together with the limited amount of fluorosurfactant (1-2 wt %) in the oil phase to synergistically stabilize the large microdroplets for PCR cycling. The positive droplets are discernible by eye, after 30-40 cycles of PCR, with a filtered transilluminator. The detection limits are shown to be 3.8 (N target region) and 3.0 (ORF1ab target region) copies per 20 μL PCR reaction mixture (or 10 μL of RNA sample). The dynamic range is 4 to 100 copies and the assay time is less than 1.5 hours.

### Methods of Manufacture

Pipette tips described herein may be manufactured by an injection molding process. Also described herein is a mold for manufacturing a pipette tip by an injection mold process. In some embodiments, pipette tips are injection molded as a unitary construct. Injection molding is a manufacturing process for producing objects (e.g., pipette tips) from thermoplastic (e.g., nylon, polypropylene, polyethylene, polystyrene and the like) and thermosetting plastic (e.g., epoxy and phenolics) materials. The plastic material of choice often is fed into a heated barrel, mixed, and forced into a mold cavity where it cools and hardens to the configuration of the mold cavity. The melted or molten material may be forced or injected into the mold cavity, through openings (e.g., a sprue), under pressure. A pressure injection method ensures the complete filling of the mold with the melted plastic. After the mold cools, the mold portions are separated, and the molded object is ejected. In some embodiments, additional additives can be included in the plastic or heated barrel to give the final product additional properties (e.g., anti-microbial, anti-static properties).

The mold is configured to hold the molten plastic or polymer in the mold cavity in the correct geometry to yield the desired object (e.g., pipette tip) upon cooling and solidification of the plastic or polymer. Injection molds may be made of two or more parts and comprise a core pin. The core pin can determine the thickness of the object wall, as the distance between the core pin and the outer mold portion is the wall thickness. Molds are typically designed so that the molded part reliably remains on the core pin when the mold opens, after cooling. The core pin sometimes can be referred to as the ejector side of the mold. The part can then fall freely away from the mold when ejected from the core pin, or ejector side of the mold. In some embodiments, the pipette tip injection mold comprises a geometry that is configured to produce a pipette tip having an orifice aspect ratio of equal to or greater than about 3.5.

Pipette tips described herein may be manufactured from a commercially suitable material. Pipette tips often are manufactured from one or more moldable materials, independently selected from those that include, without limitation, polypropylene (PP), polyethylene (PE), high-density polyethylene (HDPE), low-density polyethylene (LDPE), polyethylene terephthalate (PET), polyvinyl chloride (PVC), polytetrafluoroethylene (PTFE), polystyrene (PS), high-density polystyrene, acrylonitirle butadiene styrene copolymers, crosslinked polysiloxanes, polyurethanes, (meth)acrylate-based polymers, cellulose and cellulose derivatives, polycarbonates, ABS, tetrafluoroethylene polymers, corresponding copolymers, plastics with higher flow and lower viscosity or a combination of two or more of the foregoing, and the like.

Described herein is a method for manufacturing a pipette tip having an orifice aspect ratio of equal to or greater than about 3.5, the method comprising: providing an injection mold comprising a mold cavity having a geometry configured to produce a pipette tip having an orifice aspect ratio of equal to or greater than about 3.5; contacting the mold cavity with a molten polymer; incubating the molten polymer in the mold cavity for sufficient time to allow the molten polymer to cool and solidify in the mold cavity; and releasing the formed pipette tip from the mold cavity after cooling. Another aspect is a pipette tip having an orifice aspect ratio of equal to or greater than about 3.5 produced by the foregoing method.

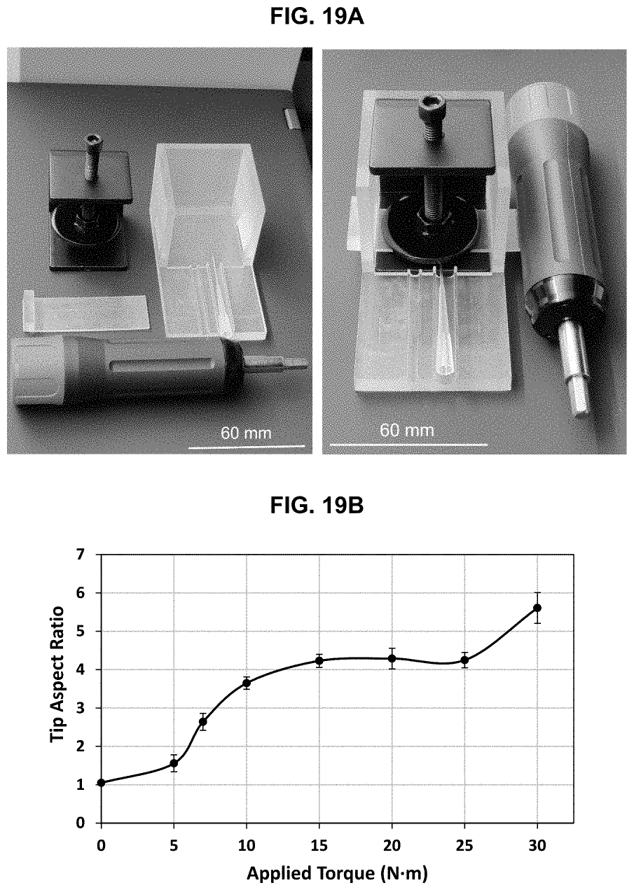

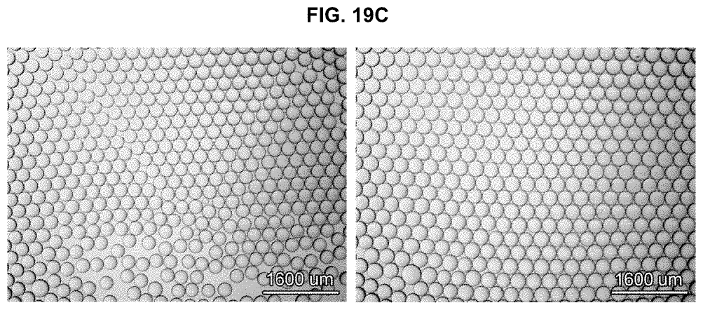

Also described herein is a method for manufacturing a pipette tip having an orifice aspect ratio of equal to or greater than about 3.5 using a commercially available pipette tip, the method comprising: selecting a commercially available pipette tip; and deforming the pipette tip orifice circumference to have an orifice aspect ratio of equal to or greater than about 3.5. In one aspect of the invention, in order to standardize the modification of the commercial pipette tips to prepare head-flattened tips at point-of-need, specific pressures can be transferred from a torque screwdriver to deform the round orifices of the commercial pipette tips to elliptical orifices of desired aspect ratios for droplet generation, as demonstrated in FIG. 19A-C. In another aspect, the deforming step comprises applying sufficient pressure between two surfaces to a longitudinal section of the pipette tip adjacent to and comprising the tip orifice for sufficient time to deform the orifice circumference to have an orifice aspect ratio of equal to or greater than about 3.5. Another aspect is a modified pipette tip having an orifice aspect ratio of equal to or greater than about 3.5 produced by the foregoing method.

### Methods for Generating Droplets and PCR

Another embodiment described herein is a method of rapidly generating a plurality of large uniform droplets for performing droplet digital Polymerase Chain Reaction (ddPCR) assays, the method comprising: (a) providing a pipette affixed to a pipette tip having a tip orifice aspect ratio of equal to or greater than about 3.5 and a surfactant coating on an interior axial bore surface of the pipette tip; (b) loading the pipette tip with a PCR reaction mixture; and (c) continuously expelling the PCR reaction mixture from the pipette tip, thereby generating a plurality of large uniform droplets. In one aspect, the pipette tip having an orifice aspect ratio of equal to or greater than about 3.5 is generated by: providing a commercially available pipette tip; and applying sufficient pressure between two surfaces to a longitudinal section of the pipette tip adjacent to and comprising the tip orifice for sufficient time to deform the orifice circumference to have an orifice aspect ratio of equal to or greater than about 3.5. In another aspect, the surfactant coating on the interior axial bore of the pipette tip is prepared by contacting the interior axial bore surface of the pipette tip with one or more surfactants, incubating the surfactant for a sufficient period of time to adhere to the interior axial bore surface; and expelling the surfactant.

Another embodiment described herein is a method of rapidly generating a plurality of large uniform droplets for performing droplet digital Polymerase Chain Reaction (ddPCR) assays, the method comprising: (a) providing a commercially available pipette tip; (b) deforming the pipette tip orifice circumference to have an orifice aspect ratio of equal to or greater than about 3.5; (c) contacting an interior sidewall of an interior axial bore of the pipette tip with one or more surfactants to generate a surfactant coating; (d) loading the pipette tip having an orifice aspect ratio of equal to or greater than about 3.5 and a surfactant coating with a PCR reaction mixture; and (e) continuously expelling the PCR reaction mixture from the pipette tip, thereby generating a plurality of large uniform droplets. In one aspect, deforming the pipette tip orifice circumference comprises applying sufficient pressure between two surfaces to a longitudinal section of the pipette tip adjacent to and comprising the tip orifice for sufficient time to deform the orifice circumference to have an orifice aspect ratio of equal to or greater than about 3.5. In another aspect, the one or more surfactants comprises a fluorosurfactant in an oil phase of the pristine oil HFE-7500 Engineered Fluid (3M Novec). In another aspect, the fluorosurfactant comprises RAN-008, Krytox, or combinations thereof. In another aspect, the fluorosurfactant is present in the oil phase at a concentration ranging from about 1 w/v % to about 5 w/v %. In another aspect, the method further comprises a water-soluble surfactant in an aqueous phase to stabilize the droplets in the oil phase during the ddPCR assay. In another aspect, the water-soluble surfactant is polyoxyalkylene block copolymer surfactant (Pluronic F127 or Poloxamer 188). In another aspect, the polyoxyalkylene block copolymer surfactant (Pluronic F127 or Poloxamer 188) is present in the aqueous phase at a concentration of 0.01 w/v %. In another aspect, the polyoxyalkylene block copolymer surfactant (Pluronic F127 or Poloxamer 188) is present in the aqueous phase at a concentration ranging from about 0.01 w/v % to about 1.5 w/v %. In another aspect, the plurality of large uniform droplets comprises individual droplets having a size ranging from about 100 μm to about 400 μm. In another aspect, the plurality of large uniform droplets comprises individual droplets having a size ranging from about 150 μm to about 350 μm. In another aspect, the plurality of large uniform droplets comprises individual droplets having a size ranging from about 150 μm to about 200 μm. In another aspect, the plurality of large uniform droplets comprises between about 1,000 to about 10,000 monodispersed droplets. In another aspect, the plurality of large uniform droplets comprises about 5,000 monodispersed droplets. In another aspect, the plurality of large uniform droplets is generated in less than 5 min. In another aspect, the plurality of large uniform droplets has a coefficient of variation (CV) of less than 3%. In another aspect, the plurality of large uniform droplets has a CV of about 2.3%. In another aspect, the plurality of large uniform droplets is visually analyzed following the ddPCR assay using a camera or microscope. In another aspect, the pipette tip has an orifice aspect ratio ranging from about 3.5 to about 6. In another aspect, the pipette tip has an orifice aspect ratio ranging from about 3.5 to about 5. In another aspect, the method accelerates the total ddPCR assay processing time to less than about 1.5 hr. In another aspect, the method has a limit of detection (LoD) of 3.8 copies per 20 μL reaction. In another aspect, the method has a dynamic range of 4 to 100 copies. In another aspect, the method is performed outside of a centralized lab and is suitable for point-of-care (POC) quantification applications.

### Methods for Detection

Another embodiment described herein is a method of detecting or measuring the presence of a virus in a subject by rapidly generating a plurality of large uniform droplets for performing rapid droplet digital Polymerase Chain Reaction (ddPCR) assays, the method comprising: (a) obtaining a sample from the subject; (b) processing the sample for ddPCR analysis; (c) loading a pipette tip having an orifice aspect ratio of equal to or greater than about 3.5 and a surfactant coating with a PCR reaction mixture containing the processed sample; (d) continuously expelling the PCR reaction mixture from the pipette tip, thereby generating a plurality of large uniform droplets; (e) performing the ddPCR assay; and (f) visually analyzing the plurality of droplets following the ddPCR assay to detect or measure the presence of a virus in the sample. In one aspect, the sample comprises whole blood, serum, plasma, urine, tears, sweat, saliva, nasopharyngeal fluid, lymph, cerebrospinal fluid, fecal extract, cellular or tissue extracts, or any other aqueous sample. In another aspect, the virus is SARS-CoV-2. In another aspect, the plurality of droplets is visually analyzed using a camera or microscope.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A pipette tip for performing rapid droplet digital Polymerase Chain Reaction (ddPCR) assays, the pipette tip comprising a proximal end having a first orifice adapted to be operably connected to a pipette, an elongated main body having a cylindrical sidewall forming an interior axial bore and tapering longitudinally from the proximal end to a distal end having a second orifice, the second orifice being in communication with the interior axial bore and adapted to allow the passage of fluid therethrough, and the second orifice having an elliptical cross-section with an orifice aspect ratio greater than about 3.5.
Clause 2. The pipette tip of clause 1, wherein the second orifice has an orifice aspect ratio ranging from about 3.5 to about 6.
Clause 3. The pipette tip of clause 1 or 2, wherein the second orifice has an orifice aspect ratio ranging from about 3.5 to about 5.
Clause 4. The pipette tip of any one of clauses 1-3, wherein an interior sidewall of the interior axial bore is coated with one or more surfactants.
Clause 5. The pipette tip of any one of clauses 1-4, wherein the one or more surfactants comprises a fluorosurfactant in an oil phase.
Clause 6. The pipette tip of any one of clauses 1-5, wherein the one or more surfactants comprises a water-soluble surfactant in an aqueous phase.
Clause 7. A method for modifying a commercially available pipette tip, the method comprising:
   (a) selecting a commercially available pipette tip; and
   (b) deforming the pipette tip orifice circumference to have an orifice aspect ratio of equal to or greater than about 3.5.
Clause 8. A modified pipette tip produced by the method of clause 7.
Clause 9. A method for manufacturing a pipette tip having an orifice aspect ratio of equal to or greater than about 3.5, the method comprising:
   (a) providing an injection mold comprising a mold cavity having a geometry configured to generate a pipette tip having an orifice aspect ratio of equal to or greater than about 3.5;
   (b) contacting the mold cavity with a molten polymer;
   (c) incubating the molten polymer in the mold cavity for sufficient time to allow the molten polymer to cool and solidify in the mold cavity; and
   (d) releasing the formed pipette tip from the mold cavity after cooling.
Clause 10. A pipette tip produced by the method of clause 9.
Clause 11. A method of rapidly generating a plurality of large uniform droplets for performing droplet digital Polymerase Chain Reaction (ddPCR) assays, the method comprising:
   (a) providing a pipette affixed to a pipette tip having a tip orifice aspect ratio of equal to or greater than about 3.5 and a surfactant coating on an interior axial bore surface of the pipette tip;
   (b) loading the pipette tip with a PCR reaction mixture; and
   (c) continuously expelling the PCR reaction mixture from the pipette tip, thereby generating a plurality of large uniform droplets.
Clause 12. The method of clause 11, wherein the pipette tip having an orifice aspect ratio of equal to or greater than about 3.5 is generated by:
   providing a commercially available pipette tip; and
   applying sufficient pressure between two surfaces to a longitudinal section of the pipette tip adjacent to and comprising the tip orifice for sufficient time to deform the orifice circumference to have an orifice aspect ratio of equal to or greater than about 3.5.
Clause 13. The method of clause 11 or 12, wherein the surfactant coating on the interior axial bore of the pipette tip is prepared by contacting the interior axial bore surface of the pipette tip with one or more surfactants, incubating the surfactant for a sufficient period of time to adhere to the interior axial bore surface; and expelling the surfactant.
Clause 14. A method of rapidly generating a plurality of large uniform droplets for performing droplet digital Polymerase Chain Reaction (ddPCR) assays, the method comprising:
   (a) providing a commercially available pipette tip;
   (b) deforming the pipette tip orifice circumference to have an orifice aspect ratio of equal to or greater than about 3.5;
   (c) contacting an interior sidewall of an interior axial bore of the pipette tip with one or more surfactants to generate a surfactant coating;
   (d) loading the pipette tip having an orifice aspect ratio of equal to or greater than about 3.5 and a surfactant coating with a PCR reaction mixture; and
   (e) continuously expelling the PCR reaction mixture from the pipette tip, thereby generating a plurality of large uniform droplets.
Clause 15. The method of clause 14, wherein deforming the pipette tip orifice circumference comprises applying sufficient pressure between two surfaces to a longitudinal section of the pipette tip adjacent to and comprising the tip orifice for sufficient time to deform the orifice circumference to have an orifice aspect ratio of equal to or greater than about 3.5.
Clause 16. The method of clause 14 or 15, wherein the one or more surfactants comprises a fluorosurfactant in an oil phase.
Clause 17. The method of any one of clauses 14-16, wherein the fluorosurfactant comprises RAN-008, Krytox, or a combination thereof, in the oil phase of HFE-7500 oil.
Clause 18. The method of any one of clauses 14-17, wherein the fluorosurfactant is present in the oil phase at a concentration ranging from about 1 w/v % to about 5 w/v %.
Clause 19. The method of any one of clauses 14-18, further comprising a water-soluble surfactant in an aqueous phase to stabilize the droplets in the oil phase during the ddPCR assay.
Clause 20. The method of any one of clauses 14-19, wherein the water-soluble surfactant is polyoxyalkylene block copolymer surfactant Pluronic F127, Poloxamer 188, or a combination thereof.
Clause 21. The method of any one of clauses 14-20, wherein the polyoxyalkylene block copolymer surfactant is present in the aqueous phase at a concentration of 0.01 w/v %.
Clause 22. The method of any one of clauses 14-21, wherein the polyoxyalkylene block copolymer surfactant is present in the aqueous phase at a concentration ranging from about 0.01 w/v % to about 1.5 w/v %.
Clause 23. The method of any one of clauses 14-22, wherein the plurality of large uniform droplets comprises individual droplets having a size ranging from about 100 μm to about 400 μm.
Clause 24. The method of any one of clauses 14-23, wherein the plurality of large uniform droplets comprises individual droplets having a size ranging from about 150 μm to about 350 μm.
Clause 25. The method of any one of clauses 14-24, wherein the plurality of large uniform droplets comprises individual droplets having a size ranging from about 150 μm to about 200 μm.
Clause 26. The method of any one of clauses 14-25, wherein the plurality of large uniform droplets comprises between about 1,000 to about 10,000 monodispersed droplets.
Clause 27. The method of any one of clauses 14-26, wherein the plurality of large uniform droplets comprises about 5,000 monodispersed droplets.
Clause 28. The method of any one of clauses 14-27, wherein the plurality of large uniform droplets is generated in less than 5 min.
Clause 29. The method of any one of clauses 14-28, wherein the plurality of large uniform droplets has a coefficient of variation (CV) of less than 3%.
Clause 30. The method of any one of clauses 14-29, wherein the plurality of large uniform droplets has a CV of about 2.3%.
Clause 31. The method of any one of clauses 14-30, wherein the plurality of droplets is visually analyzed following the ddPCR assay using a camera or microscope.
Clause 32. The method of any one of clauses 14-31, wherein the pipette tip has an orifice aspect ratio ranging from about 3.5 to about 6.
Clause 33. The method of any one of clauses 14-32, wherein the pipette tip has an orifice aspect ratio ranging from about 3.5 to about 5.
Clause 34. The method of any one of clauses 14-33, wherein the method accelerates the total ddPCR assay processing time to less than about 1.5 hr.
Clause 35. The method of any one of clauses 14-34, wherein the method has a limit of detection (LoD) of 3.8 copies per 20 μL reaction.
Clause 36. The method of any one of clauses 14-35, wherein the method has a dynamic range of 4 to 100 copies.
Clause 37. The method of any one of clauses 14-36, wherein the method is performed outside of a centralized lab and is suitable for point-of-care (POC) quantification applications.
Clause 38. A method of detecting or measuring the presence of a virus in a subject by rapidly generating a plurality of large uniform droplets for performing rapid droplet digital Polymerase Chain Reaction (ddPCR) assays, the method comprising:
   (a) obtaining a sample from the subject;
   (b) processing the sample for ddPCR analysis;
   (c) loading a pipette tip having an orifice aspect ratio of equal to or greater than about 3.5 and a surfactant coating with a PCR reaction mixture containing the processed sample;
   (d) continuously expelling the PCR reaction mixture from the pipette tip, thereby generating a plurality of large uniform droplets;
   (e) performing the ddPCR assay; and
   (f) visually analyzing the plurality of droplets following the ddPCR assay to detect or measure the presence of a virus in the sample.
Clause 39. The method of clause 38, wherein the sample comprises whole blood, serum, plasma, urine, tears, sweat, saliva, nasopharyngeal fluid, lymph, cerebrospinal fluid, fecal extract, cellular or tissue extracts, or any other aqueous sample.
Clause 40. The method of clause 38 or 39, wherein the virus is SARS-CoV-2.
Clause 41. The method of any one of clauses 38-40, wherein the plurality of large uniform droplets is visually analyzed using a camera or microscope.

### EXAMPLES

### Example 1

### PCR Reaction

A typical 20 μL reaction mixture was prepared by mixing 5 μL Master Mix (4×, TaqPath™ 1-Step Multiplex Master Mix (Thermo ScientificTM)), 1 μL 2019-nCoV assay (20×, TaqMann™ 2019nCoV Assay Kit v1 (Thermo ScientificTM)), 2 μL 20 wt % Pluronic® F127 (Sigma-Aldrich, BioReagent) aqueous solution, 10 μL template sample and 2 μL nuclease-free water. The 1 μL 2019-nCoV reverse-transcription PCR assay consisted of N primers/probe sets or ORF1ab primers/probe set. The volumes of Pluronic® F127 solution, template sample and nuclease-free water could be adjusted according to template concentration and droplet size for optimum efficiency and stability. After reaction mixture preparation, droplets were generated in 200 μL PCR tubes as described below.

### Modification of Commercialy Available Pipette Tip

The tip region of a commercially available pipette tip (e.g., Eppendorf microloader) was modified (e.g., flattened) by pressing the tip between a glass slide and a lab bench for 10 seconds to deform the distal end adjacent to and comprising the tip orifice to achieve an orifice aspect ratio of equal to or greater than about 3.5.

### Droplet Generation

After modifying the pipette tip, 10 μL of oil (HFE-7500; 3M Novec with 1-2 wt % RAN 008 surfactant, or with 5 wt % Krytox; see Table 1) was drawn into the modified pipette tip using a standard pipette to coat the interior of the modified pipette tip with the surfactant. The oil was expelled out and 20 μL of the PCR reaction mixture was immediately drawn into the modified pipette tip. Subsequently, the PCR reaction mixture was pipetted into 50 μL of oil (HFE-7500; 3M Novec with 1-2 wt % RAN 008 surfactant, or with 5 wt % Krytox; see Table 1) in a 200 μL PCR tube. Multiple droplets (i.e., a plurality of large uniform droplets) were generated by continuously expelling the PCR reaction mixture from the modified pipette tip, where each actuation generated one or more monodispersed droplets. This process took 3-5 minutes to convert the entire 20 μL PCR reaction into ˜5000 droplets. Exemplary surfactants are shown below in Table 1.

TABLE 1
Surfactants

| Reagent | Vendor | Formula/Abbreviation | Chemical Composition |
|---|---|---|---|
| RAN-008- | RAN Biotechnologies | PFPE- | CF₃—(CF₂)₂—O—(CF—(CH₃)—CF₂—O)ₙ— |
| FluoroSurfactant | | PEG/PEO- | CF(CF₃)—CO—NH—(CH₂CH₂O)ₘ— |
| | | PFPE^{[1]} | CH₂CH₂—NH—CO—CF(CH₃)ₙ—(O— |
| | | | CF₂CF(CF₃)—O—(CF₂)₂)—CF₃ |
| Krytox ® 157 FSH | DuPont | PFPE-COOH^{[2]} | F—(CF(CF₃)—CF₂—O)ₙ—CF(COOH)CF₃ |
| Pluronic ® F127 | Sigma-Aldrich | PPO-PEO- | H(OCH₂CH₂)ₓ—(OCH₂CH(CH₃)H)_{y}— |
| | | PPO^{[3]} | (OCH₂CH₂)_{z}—OH |

^{[x]} refers to specific formulations discussed in the specification above.

### Droplet Digital PCR

After generating droplets, the PCR tubes were placed into a thermal cycler (Bio-Rad, MJ Mini Thermal Cycler) and thermal cycling was performed at 50° C. for 5 min, 95° C. for 2 min and then 30-40 cycles of 95° C. for 3 s, 60° C. for 30 s. Including the heating/cooling times for each step, typical times for reaction is 55 min for 30 cycles and 70 min for 40 cycles for this thermal cycler.

### Imaging

After PCR thermal cycling, the droplets together with the oil in the 200 μL tube were collected and transferred to the center of a covered frame seal on a glass slide. The entire monolayer suspension occupies an area of about 1 cm² and hence easily fit into visual or smartphone field of view such that one image contains all the droplets. As a reference, these droplets were imaged by a table-top fluorescence microscope (Olympus IX71) or a portable fluorescence mini-microscope (Dino-Lite™ Edge AM4115T-GFBW). For smartphone camera imaging or visual counting, the glass slide with droplets were placed onto a transilluminator (Clare Chemical Research) and the blue light illuminated droplets in dark or dim ambient environment were recorded by the camera of a OnePlus 7 Pro smartphone.

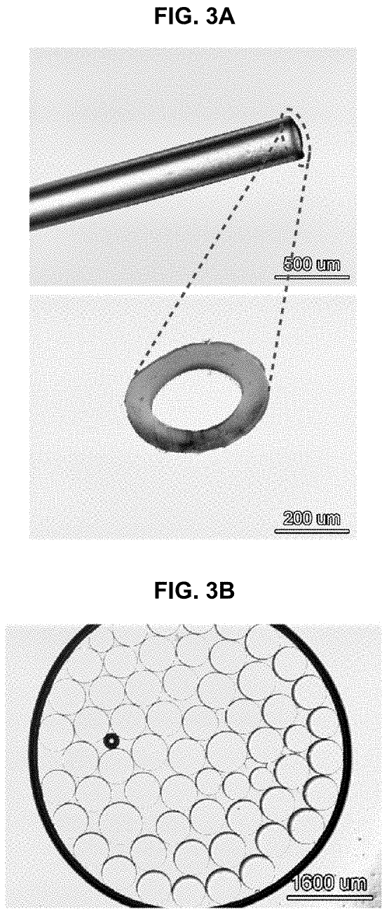

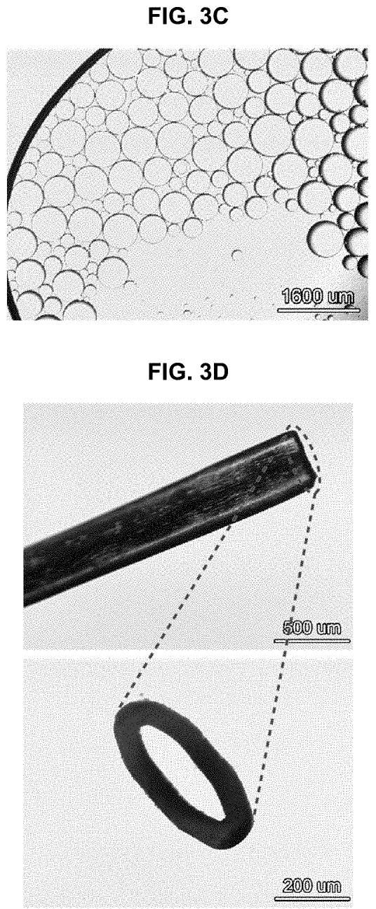

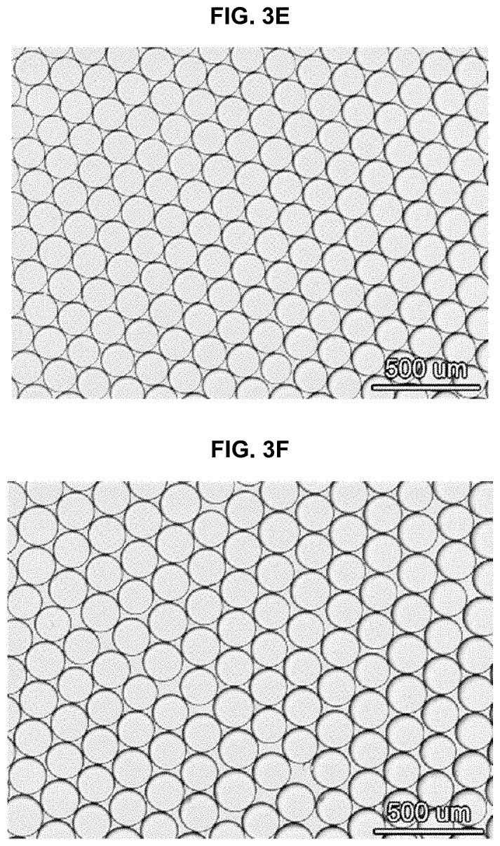

As a reference, a commercial micro-loading pipette tip (˜200 μm diameter orifice) was first used to generate water droplets in HFE-7500 oil with 2 wt % fluorosurfactant (FIG. 3A). The prepared water droplets were mostly around 600 μm in diameter and the droplets showed significant CV of ˜9.6% in the droplet size (FIG. 3B). After the pipette tip was flattened to an elliptical shape with an aspect ratio of ˜4, larger than the theoretical limit of 3.5 for monodispersed droplets, uniform water droplets of 157±4 μm diameter were generated with a CV of 2.3% (FIG. 3D-E). The same modified pipette tip generated equally monodispersed but slightly larger droplets with the more viscous PCR solution (FIG. 3F, 177±6 μm diameter due to the viscosity difference, CV of 3.1%) while the unmodified tip totally failed (FIG. 3C).

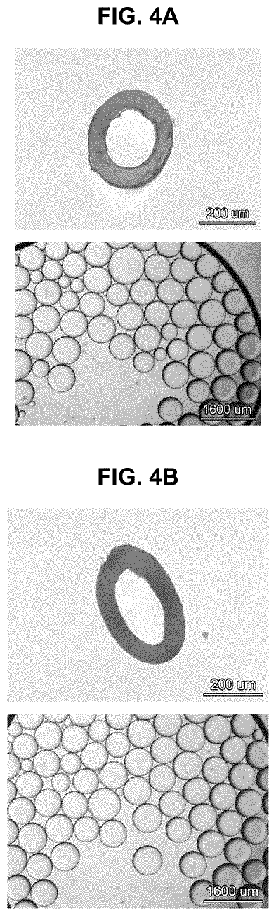

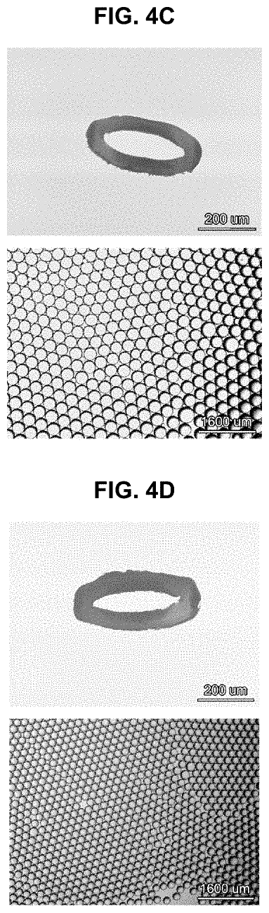

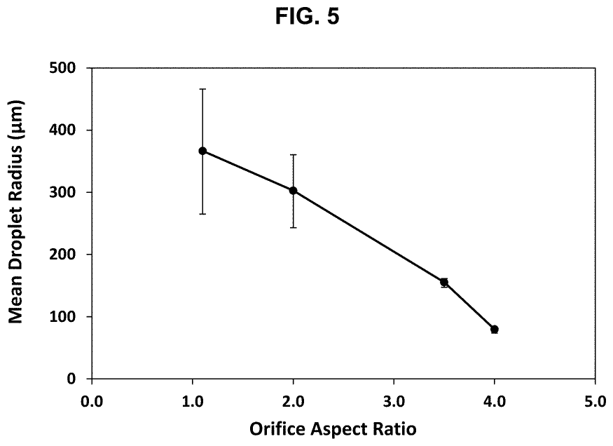

Multiple droplet generation trials with modified (tip-flattened) pipette tips of various cross-section aspect ratios were conducted. Uniform droplets were produced with aspect ratios greater than 3.5. Higher aspect ratios (such as 4-5) produced smaller and more uniform droplets (FIG. 4-5). Like their microfluidic chip counterparts, the size and monodispersity of the droplets generated by the flattened elliptical pipette tip were insensitive to the flow rate so that it was possible to generate a plurality of uniform droplets just by continuously expelling the entire PCR reaction mixture using a regular manual pipette equipped with a modified pipette tip. Usually, 3-5 minutes completed the entire -5000 droplet generation process from 20 μL PCR reaction mixture directly in a 200 μL PCR tube loaded with 50 μL oil, corresponding to a droplet generation rate of about 30 Hz. This was slow compared to commercial droplet generation technologies, but there were (100×) fewer droplets to generate. There was almost no sample loss during droplet generation and the nascent droplets created in the PCR tubes were ready for use in a thermal cycler. Pipette droplet generation directly in the PCR tube is an efficient and effective way to prepare large monodisperse droplets for ddPCR.

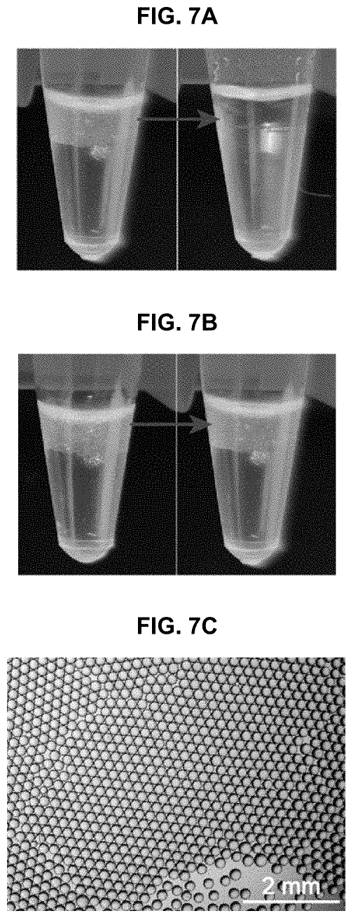

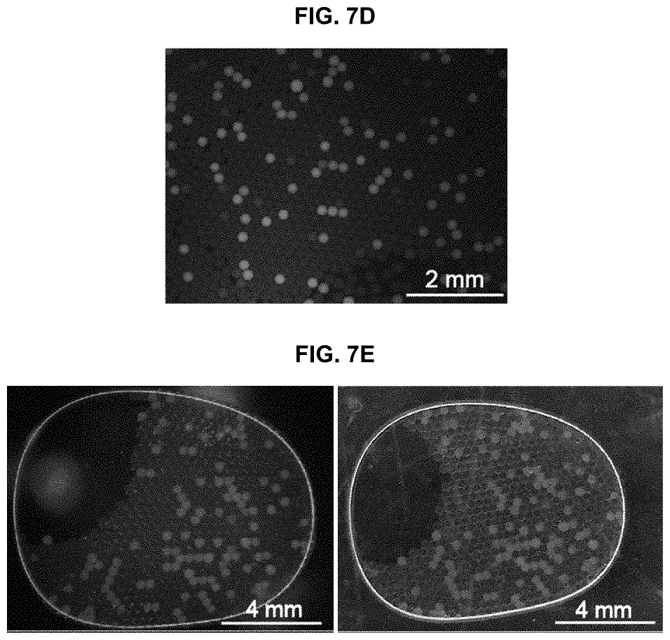

Now that uniform large microdroplets could be prepared by head-flattened tips with a pipette gun, the next step was to confirm the effectiveness of PCR reaction in the droplets. Unfortunately, after 40 PCR cycles, the large microdroplets of reaction mixture coalesced severely (FIG. 7A, right). The commercial fluorosurfactant that consisted of poly(ethylene glycol) and perfluoropolyethers (PFPE) blocks could not stabilize droplets of ˜100 μm diameter for the PCR reaction and would fare even worse for larger (>150 μm) droplets. To solve this problem, polyoxyalkylene block copolymer F127 solution was introduced into the PCR reaction mixture to stabilize the droplets from inside. The F127 three-block copolymer was chosen because it contained the polyethylene oxide (PEO) blocks, which were the hydrophilic segments of the surfactant in the oil. Another reason was that the F127 solution could respond to high temperatures with versatile micelle formation and aggregation/patterning, which was the mechanism for the gelation of F127 solution at elevated temperatures. Moreover, the polyoxyalkylene chemistry of F127 was benign to the PCR reaction. Notably, the polyoxyalkylene block copolymer Poloxamer 188 (P188) can also be used instead of F127, as they both have been found to provide sufficient droplet stabilization.

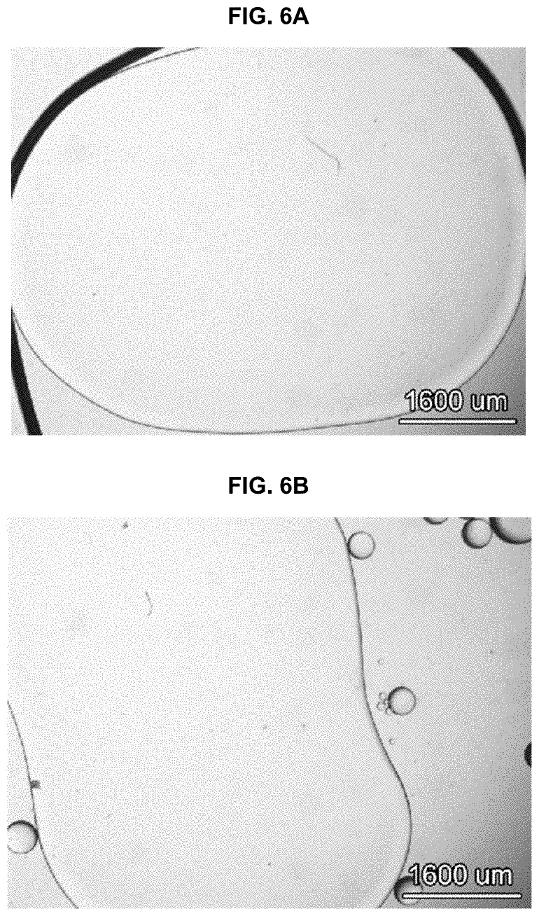

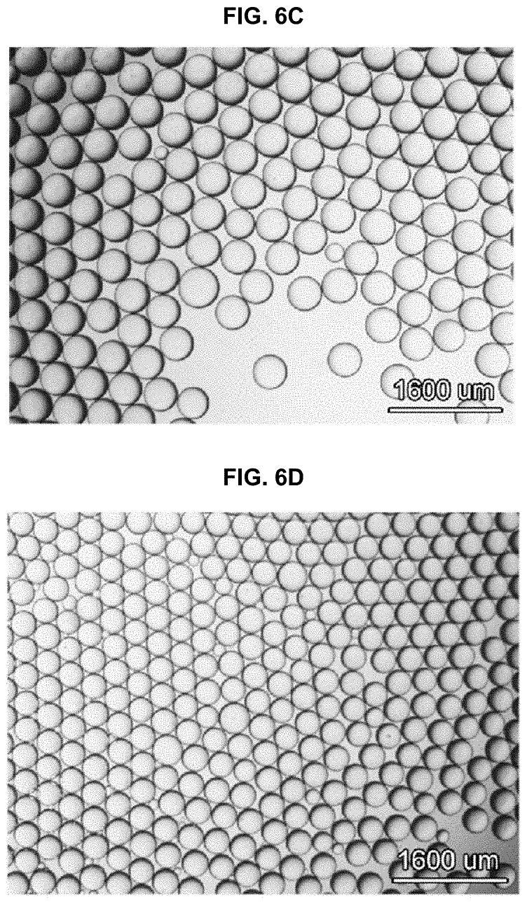

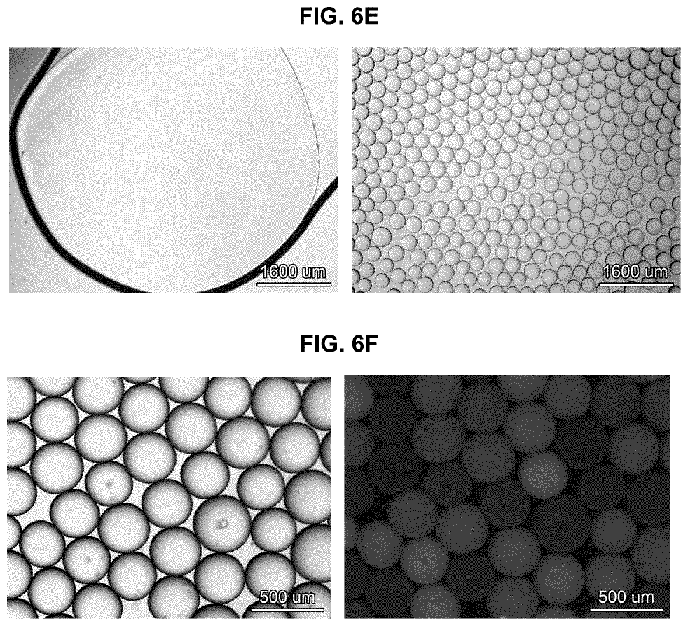

With over 0.01 w/v % of F127 in the PCR reaction mixture, the large microdroplets could withstand 40 PCR thermal cycles without droplet coalescence (FIG. 7B, right). The stabilization of aqueous droplets by F127 in fluorocarbon oil was further confirmed by generating droplets in HFE-7500 oil with 2 v/v % Krytox 157 FSH and adding increasing concentrations of either F127 or P188 (FIG. 6A-F). With over 0.01 w/v % F127 in water, uniform aqueous droplets were produced (FIG. 6C-D). Otherwise, the droplets would coalesce immediately following pipetting (FIG. 6A-B). Polyoxyalkylene block copolymer P188 (0.5 wt %) helped stabilize water droplets similar to F127 (FIG. 6E).

After stabilizing the large droplets for PCR thermal cycling, it was established that large droplets of ˜150-350 μm range can produce visually or smartphone detectable droplets with a transilluminator. As a benchmark, both a table-top commercial fluorescent microscope (FIG. 7C-D) and a hand-held fluorescent microscope (FIG. 7E, left) were also used. All images by different detectors allow easy identification of positive droplets (FIG. 7D-E). Because the droplets were over 150 μm, positive droplets could also be discerned by human eyes from a distance of 50 cm, when illuminated by a universal filtered transilluminator with blue light in dark or dim ambient environment. Hence, a smartphone camera or even naked human eyes were able to record the positive droplets with good fidelity (FIG. 7E, right). The hand-held microscope had better resolution than the smartphone camera images (FIG. 7E, left) for the same droplet suspension, but the positive droplets are completely discernable in the smartphone camera image.

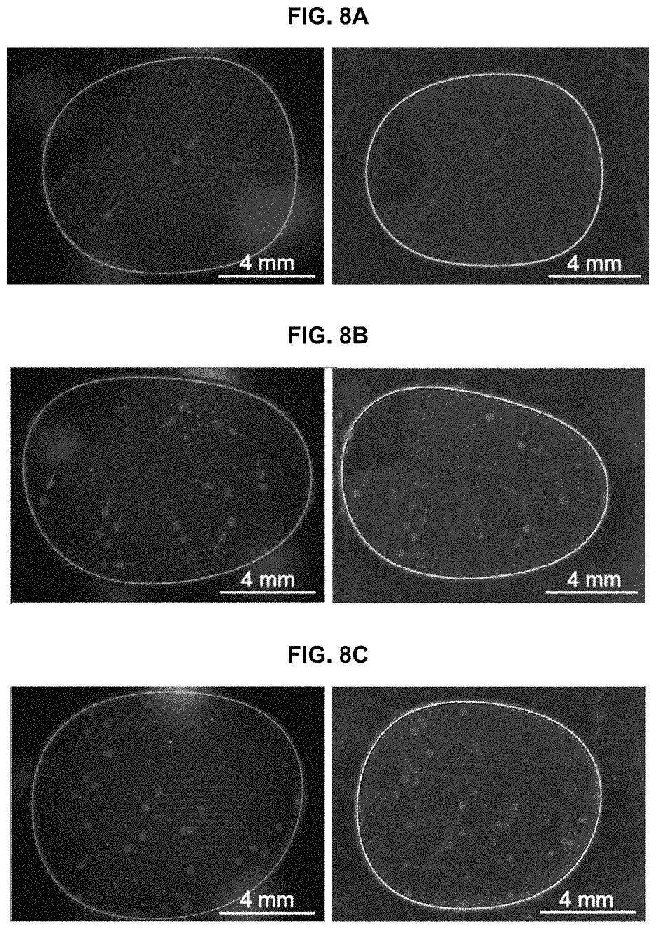

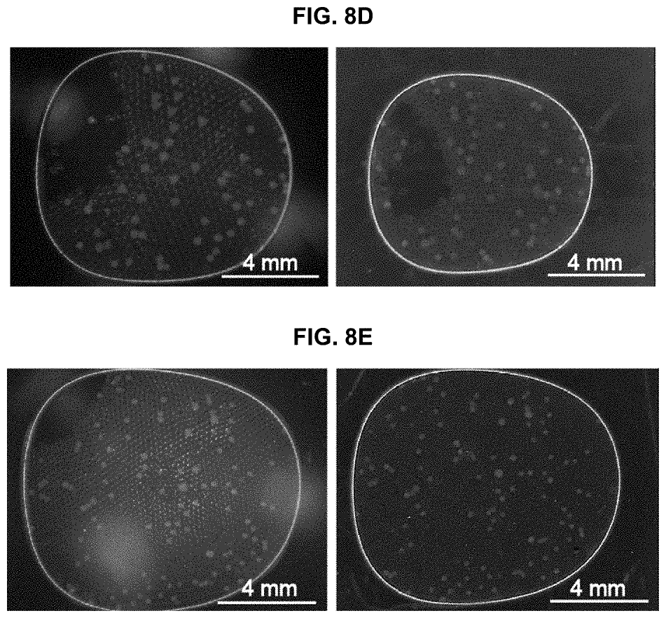

The resolution by visual detection was scrutinized. A series of 20 μL PCR reaction mixtures were prepared with nominally 2, 10, 25, 50, or 100 copies of template and two target regions (N, ORF1ab) were detected. FIG. 8 shows the results of the handheld mini-microscope and smartphone camera images of the samples, each with a 20 μL PCR reaction mixture. The smartphone camera images had the identical numbers of lit droplets to mini-microscope images and the numbers were close to the nominal values. For small copy number samples with 2, 10, 25, and 50 copies of target, droplets used were around 300 μm generated by head-flattened tips with cross-section aspect ratios slightly larger than 3.5, for better visual detection after PCR reaction. This larger droplet size produced about 1500 droplets from 20 μL of the reaction mixture. It was a large enough droplet number relative to the copy number (larger than a factor of ˜30) to ensure no more than one target per droplet so that quantification could be done by counting only the lit positive droplets.

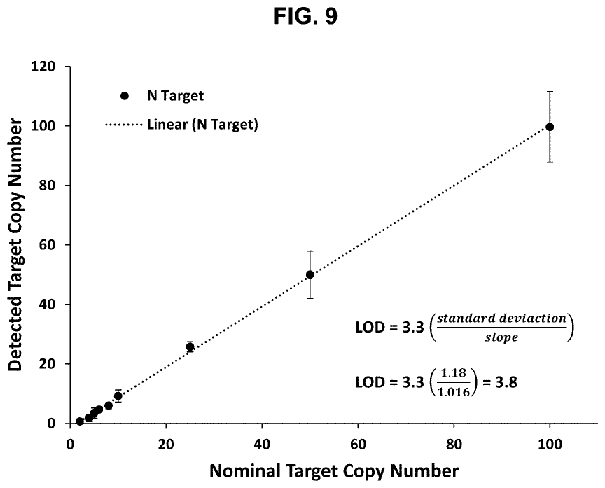

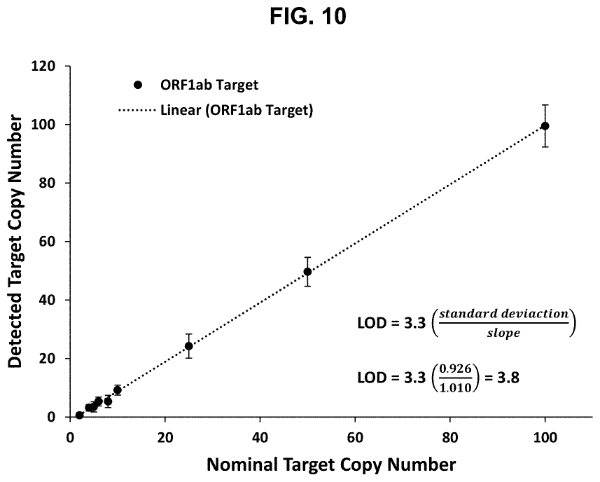

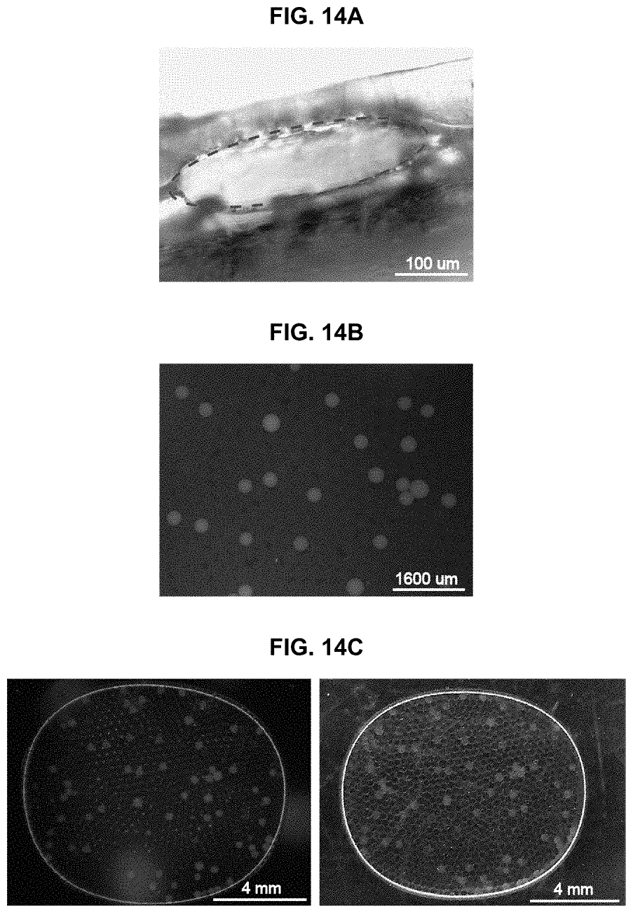

Droplets around 300 μm could also be produced by commercial gel-loading flat pipette tips for droplet digital PCR tests (FIG. 14A-C). For the higher copy number samples with nominally 100 target copies (FIG. 8E), more droplets were required, and about 5000 of 200 μm droplets were generated from 20 μL of the reaction mixture with a tip cross-section aspect ratio close to 4 to guarantee Poisson distribution of one target per positive droplet. Repeated experiments (over 5 times for each sample point) were conducted for both N and ORF1ab target sequences (FIG. 9A-B). Based on regression method with a 95% confidence interval, the estimated limit-of-detections (LoDs) were 3.8 (N target sequence) and 3.0 (ORF1ab target sequence) copies per 20 μL of the reaction (FIG. 10A-B). If mean values and standard deviations of blank samples and low concentration samples were used, the calculated LoDs were 2.2 (N target sequence) and 2.5 (ORF1ab target sequence) copies per 20 μL of reaction (Table 2). Both estimates were comparable and consistent with reported values for commercial ddPCR that use expensive optics.

From the blank sample data shown below in Table 2, contamination and primer dimerization were a source of error at low copy number. Other larger sources of error were, however, analyte loss during pipette handling, sample dilution at low target number, and insufficient droplet number at large copy number (approaching 100) to ensure a single copy per positive droplet (from the growing standard deviation with respect to copy number in FIG. 9).

TABLE 2
Calculation of the Limit-Of-Detections from Mean Values and Standard
Deviations of Blank Samples and Low-Concentration Samples

| Number of | | | | | | | | | | |
|---|---|---|---|---|---|---|---|---|---|---|
| Target(s) in | | | | | | | | | Limit | Limit |
| 20 μL PCR | | | | | | | | | of | of |
| Reaction | Number of Positive | | | | | | | Standard | Blank | Detection |
| Mixture* | Droplet(s) Detected | | | | | | Mean | Deviation | (LoB) | (LoD) |
| 0-copy N | 0 | 0 | 0 | 0 | 0 | 1 | 0.17 | 0.41 | 0.84 | 2.18 |
| 2-copy N | 0 | 0 | 1 | 1 | 0 | 2 | 0.67 | 0.82 | | |
| 0-copy ORF1ab | 0 | 0 | 1 | 0 | 1 | 0 | 0.33 | 0.52 | 1.18 | 2.53 |
| 2-copy ORF1ab | 0 | 2 | 0 | 1 | 0 | 1 | 0.67 | 0.82 | | |

*Samples with 0-copy target are blank samples and samples with 2-copy target are low concentration samples.

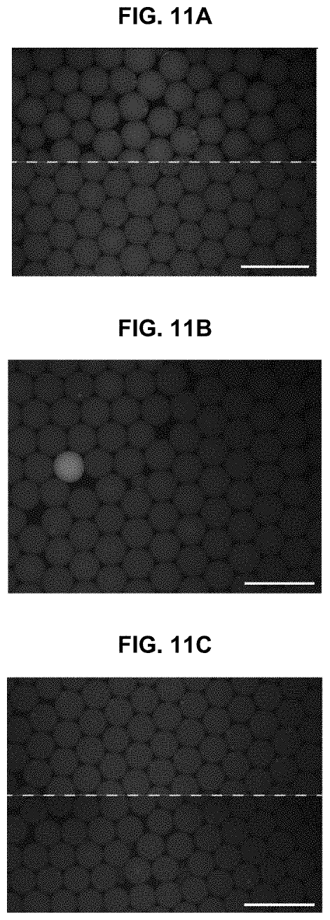

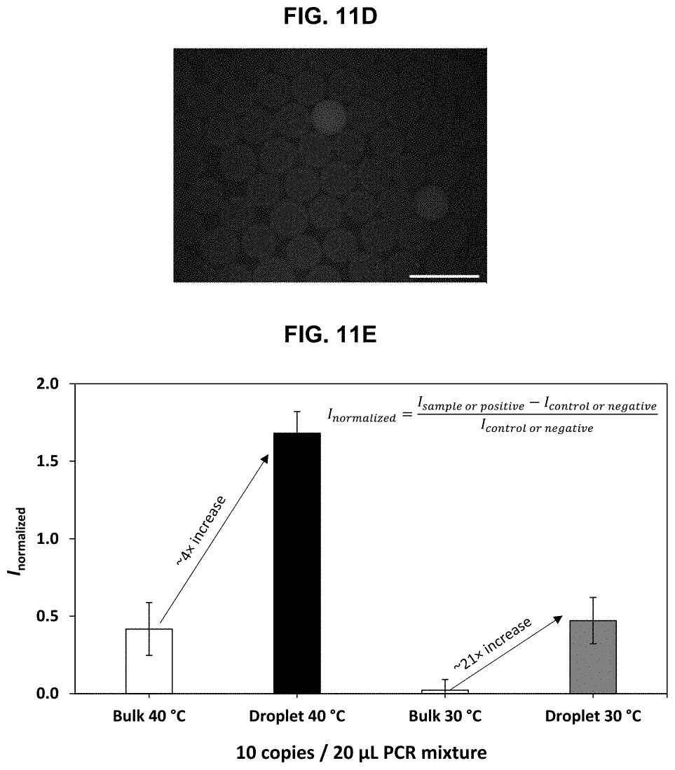

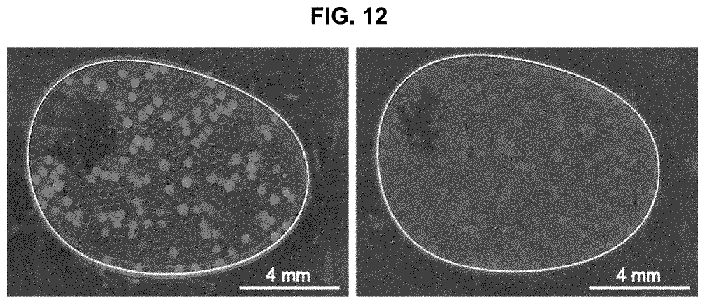

Moreover, it was confirmed that the 150-350 μm droplets allow quantification by eye after a typical 30-40 cycle amplification. Reactions with no template or 10 copies of template in 20 μL mixture were done in bulk and in droplets. After cycling, the bulk mixture was converted into droplets for fluorescence intensity comparison. The normalized fluorescence intensity differences from bulk PCR samples with 0 or 10 target copies (FIG. 11A, 11C) and from positive and negative droplets of droplet PCR (FIG. 11B, 11D) showed that PCR reaction done in droplets had better contrast by ˜21 times at the threshold of 30 cycles when the bulk intensity could not be discerned (FIG. 11E). In general, 30-40 cycles were sufficient to allow detection of positive droplets with single molecules by eye or by smartphone imaging (FIG. 12). These were high but feasible cycle numbers that could be achieved with a typical PCR mixture.

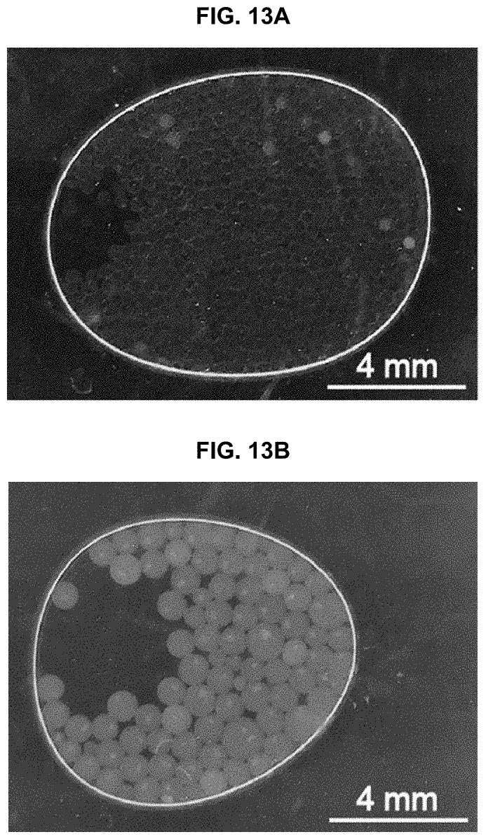

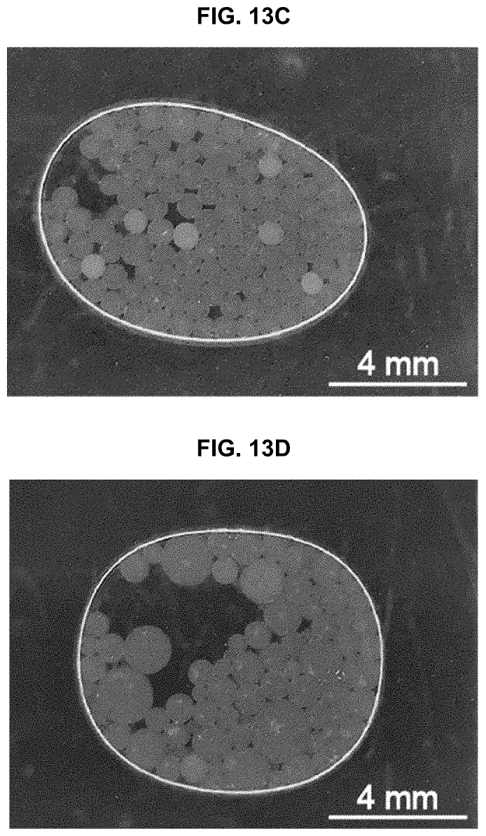

Finally, the possibility of using large droplets for RNA-extraction-free detection was demonstrated. Bulk rt-PCR tests of SARS-CoV-2 nasopharyngeal/sputum/saliva samples without RNA extraction have been reported. Uninfected raw saliva was added to the reaction mixture for droplet generation and PCR reaction. Because saliva changes the sample surface tension and viscosity, the droplets containing saliva were larger (FIG. 13A-D). No copy was detected from a 10-copy sample with 20 v/v % saliva, which also showed polydispersed droplet size. Nevertheless, for nominally 10 target copies, 9.6±2.1 and 8.7±2.5 (mean and standard deviation from 5 tests), positive droplets were observed for saliva-free and 10 v/v % saliva-doped samples, respectively. The difference was roughly consistent with errors due to target concentration fluctuation as shown in FIG. 9, suggesting that PCR inhibition from saliva samples can be eliminated with moderate dilution (FIG. 13D). Therefore, without testing a large number of samples, ddPCR showed promise for low viral load detection without RNA extraction from slightly diluted saliva samples.

Uniform large microdroplets with diameters ranging from 150 to 350 microns are generated by a regular pipette gun equipped with head-flattened pipette tips, in place of the bulky and delicate micropumps for smaller droplet generation. A polyoxyalkylene block copolymer surfactant (F127 or P188) is utilized to further stabilize these large droplets for robust PCR thermal cycling. The droplets have demonstrated good efficiency for ddPCR and the probable total assay time is about 1.5 hours including 10 min for sample preparation (commercial RNA extraction kit or RNA-extraction-free), 5 min for droplet generation, 70 min for reverse transcription plus 40 thermal cycles, and 5 min for droplet spreading and visual inspection.

Reduction of assay time to 1 hour or even 45 minutes is quite feasible with more expensive or specially designed thermal cyclers. Thanks to the large size, and also still significant reporter concentration, the droplets can be detected and, for small copy number, counted visually. It does not require any optical detector for low copy numbers, which is a major obstacle for POC applications. The LoDs of 3.8 copies per 20 μL reaction are as good as any commercial lab-bound optical PCR platform, bulk or digital. This technology hence satisfies all the necessary features for a rapid POC ddPCR test. Its thousands of droplets can allow a sufficiently large dynamic range of 4 to 100 copies that is still within the Poisson limit of a single molecule per droplet. For quantification of large copy number, it is better to utilize diluted samples to ensure accurate quantification and easy counting.

Though it is possible for the POC ddPCR to conduct tests without RNA extraction, purified samples would still permit more precise quantification and reduction of false negatives. Commercial kits are available for RNA extraction, but they may not extract the RNA at high yield or from large sample volume efficiently. Given the tremendous advantages of the downstream droplet generation and visual detection platform, a different paradigm for upstream pretreatment may be in order. One that can filter the original sample, say the VTM sample, down to 10 μL without virus loss would further enhance the sensitivity of the integrated platform to complement the rapidity and portability of the current POC detection platform.

### Example 2

Pipette Droplet Generation at High Flow Rates Via Periodical Pipetting

Pipette droplet generation via flattened pipette tips is insensitive to flow rates when they are relatively low (<˜0.25 μL/s). At higher flow rates (up to 2 μL/s), when pipette droplet generation becomes sensitive to continuous flow, periodical pipetting can be utilized to help produce more uniform droplets. Periodical pipetting at multiple flow rates produces different sized droplets having different CV values compared to continuous pipetting at the same flow rates, as shown below in Table 3.

TABLE 3
Droplets Generated from Different Pipetting Methods

| | Mean Droplet | | |
|---|---|---|---|
| Pipetting method and flow rate | Radius (μm) | CV | Std. Dev. (μm) |
| Periodical - 2 μL/s | 209.46 | 0.05 | 10.35 |
| Periodical - 10 μL/s | 231.22 | 0.32 | 73.28 |
| Continuous - 2 μL/s | 378.67 | 0.52 | 195.46 |
| Continuous - 10 μL/s | 508.45 | 0.82 | 417.42 |

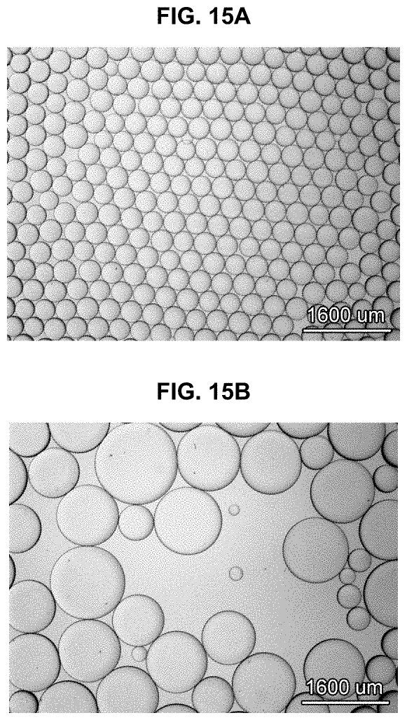

FIG. 15A shows droplets generated from periodical pipetting with a flow rate of 2 μL/s for 1 second and no flow for 2 seconds in each period. FIG. 15B shows droplets generated from continuous pipetting with a flow rate of 2 μL/s. The droplets generated by periodical pipetting are more uniform than the droplets generated by continuous pipetting. Additionally, parallel high throughput droplet generation can be realized by using a multichannel pipette gun and tips via periodical pipetting at high flow rates of ˜2 μL/s or even faster.

### Example 3

### Pipette Tip Adapter and Multi-Channel Pipette Gun for Parallel Droplet Generation

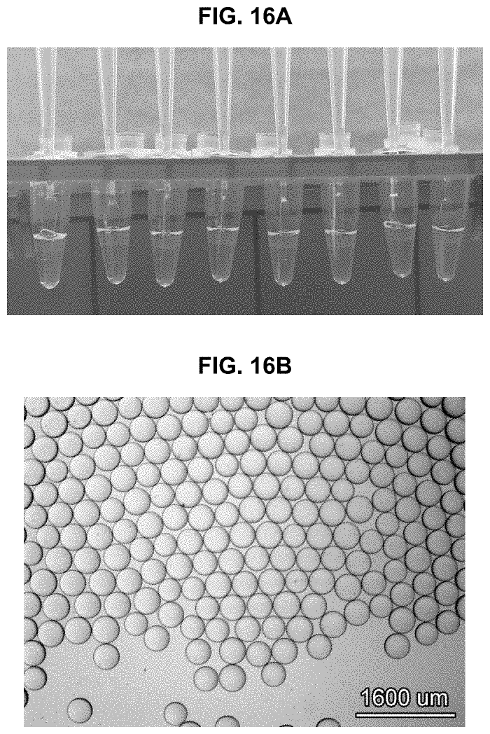

Pipette tip adapters for parallel droplet generation can be prepared using 3D printing techniques. With tip adapters, universal round-head pipette tips, and even micro-syringes, can be used for uniform droplet generation without modification. The dimension scales of the tip adapter would likely be as follows: adapter width-millimeter scale; adapter thickness-sub-millimeter to millimeter scale; and adapter channels-sub-millimeter scale with aspect ratio>3.5. Additionally, FIG. 16A-B show images of parallel droplet generation via a multi-channel pipette gun. When generating a large number of sample mixtures into uniform droplets, a multi-channel pipette gun can be used to generate droplets in parallel to improve efficiency. FIG. 16A shows an eight-channel pipette gun generating droplets inside eight PCR 200 μL tubes in parallel. FIG. 16B shows a sample of the generated droplets.

### Example 4

### Droplet Reservoir for Point-Of-Care Digital Assays

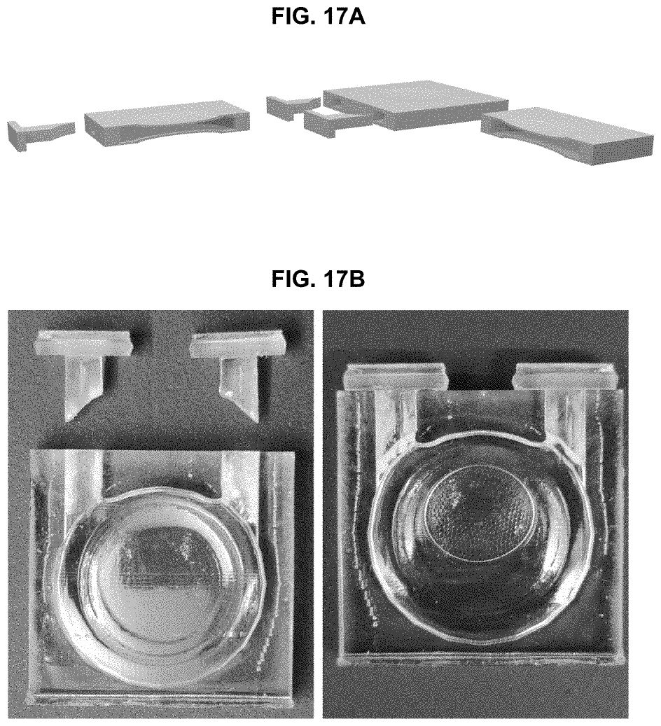

A transparent droplet reservoir for point-of-care digital assays was designed and fabricated using 3D printing techniques (FIG. 17A-B). FIG. 17A shows the 3D model of the droplet reservoir and two corresponding cross-sections. The droplet reservoir has two outlets which can be blocked by two matched plugs after droplet generation/loading. Inside the droplet reservoir, the height of the interior chamber decreases from periphery to the center so that droplets are pinned and immobilized away from the walls for easy handling and imaging. FIG. 17B shows a 3D-printed droplet reservoir with matched plugs (left) and droplets immobilized inside the droplet reservoir for imaging (right). The exterior dimensions of the droplet reservoir are 20 m×20 m×2.5 mm.

As shown in FIG. 17B (right), the droplets stay still and immobilized away from the walls inside the droplet reservoir chamber because of the changing interior chamber height. Specifically, the volume of oil containing water droplets is pipetted into the shallow imaging chamber of the reservoir, which has a height that decreases from the peripheral walls toward the chamber center. The oil wets the top and bottom chamber surfaces and forms a planar (flattened) droplet geometry in the chamber. The planar oil droplet becomes circular and immobilized at the chamber center because the chamber height is at its lowest point in the center. The periphery of this planar droplet, which is not circular or centered initially, comprises a meniscus between oil and air. As the oil wets the chamber surfaces, this meniscus resembles a semicircle that is concave outward and touches the top and bottom chamber surfaces. The air pressure remains constant inside the chamber but the oil pressure just inside the meniscus is lower by a value proportional to the inverse meniscus radius, due to the Laplace capillary effect. Hence, the oil pressure at a meniscus position closer to the chamber center will be lower than a meniscus position toward the periphery walls, and this oil pressure difference drives the oil from the periphery to the chamber center. The net result is that the periphery of the oil assumes a circular shape with a center that aligns with the chamber center, ensuring that the droplets are always centered and immobilized for easy handling and imaging.

### Example 5

### Rapid Isothermal Recombinase Polymerase Amplification (RPA) in Droplets

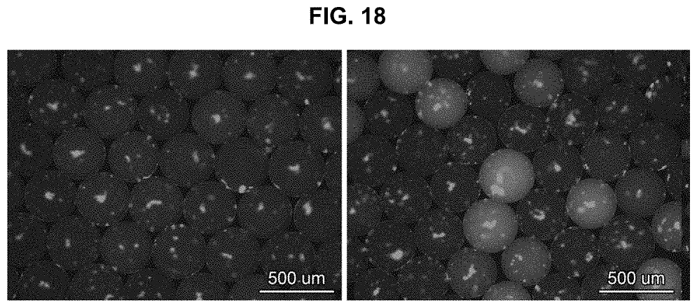

Pipette droplet generation can be performed for rapid isothermal (20 min at −40° C.) recombinase polymerase amplification (RPA). FIG. 18 shows a comparison between droplets with a blank control sample and droplets with a positive sample. Although RPA assay mixtures are intrinsically cloudy due to the formation of some precipitates, the amplification efficiency is good enough for detection after 20 min at ˜40° C.

1. A pipette tip for performing rapid droplet digital Polymerase Chain Reaction (ddPCR) assays, the pipette tip comprising a proximal end having a first orifice adapted to be operably connected to a pipette, an elongated main body having a cylindrical sidewall forming an interior axial bore and tapering longitudinally from the proximal end to a distal end having a second orifice, the second orifice being in communication with the interior axial bore and adapted to allow the passage of fluid therethrough, and the second orifice having an elliptical cross-section with an orifice aspect ratio greater than about 3.5.

2. The pipette tip of claim 1, wherein the second orifice has an orifice aspect ratio ranging from about 3.5 to about 6.

3. The pipette tip of claim 1, wherein the second orifice has an orifice aspect ratio ranging from about 3.5 to about 5.

4. The pipette tip of claim 1, wherein an interior sidewall of the interior axial bore is coated with one or more surfactants.

5. The pipette tip of claim 4, wherein the one or more surfactants comprises a fluorosurfactant in an oil phase.

6. The pipette tip of claim 4, wherein the one or more surfactants comprises a water-soluble surfactant in an aqueous phase.

7. A method for modifying a commercially available pipette tip, the method comprising:
(a) selecting a commercially available pipette tip; and
(b) deforming the pipette tip orifice circumference to have an orifice aspect ratio of equal to or greater than about 3.5.

8. A modified pipette tip produced by the method of claim 7.

9. A method for manufacturing a pipette tip having an orifice aspect ratio of equal to or greater than about 3.5, the method comprising:
(a) providing an injection mold comprising a mold cavity having a geometry configured to generate a pipette tip having an orifice aspect ratio of equal to or greater than about 3.5;
(b) contacting the mold cavity with a molten polymer;
(c) incubating the molten polymer in the mold cavity for sufficient time to allow the molten polymer to cool and solidify in the mold cavity; and
(d) releasing the formed pipette tip from the mold cavity after cooling.

10. A pipette tip produced by the method of claim 9.

11. A method of rapidly generating a plurality of large uniform droplets for performing droplet digital Polymerase Chain Reaction (ddPCR) assays, the method comprising:
(a) providing a pipette affixed to a pipette tip having a tip orifice aspect ratio of equal to or greater than about 3.5 and a surfactant coating on an interior axial bore surface of the pipette tip;
(b) loading the pipette tip with a PCR reaction mixture; and
(c) continuously expelling the PCR reaction mixture from the pipette tip, thereby generating a plurality of large uniform droplets.

12. The method of claim 11, wherein the pipette tip having an orifice aspect ratio of equal to or greater than about 3.5 is generated by:
providing a commercially available pipette tip; and
applying sufficient pressure between two surfaces to a longitudinal section of the pipette tip adjacent to and comprising the tip orifice for sufficient time to deform the orifice circumference to have an orifice aspect ratio of equal to or greater than about 3.5.

13. The method of claim 11, wherein the surfactant coating on the interior axial bore of the pipette tip is prepared by contacting the interior axial bore surface of the pipette tip with one or more surfactants, incubating the surfactant for a sufficient period of time to adhere to the interior axial bore surface; and expelling the surfactant.

14. A method of rapidly generating a plurality of large uniform droplets for performing droplet digital Polymerase Chain Reaction (ddPCR) assays, the method comprising:
(a) providing a commercially available pipette tip;
(b) deforming the pipette tip orifice circumference to have an orifice aspect ratio of equal to or greater than about 3.5;
(c) contacting an interior sidewall of an interior axial bore of the pipette tip with one or more surfactants to generate a surfactant coating;
(d) loading the pipette tip having an orifice aspect ratio of equal to or greater than about 3.5 and a surfactant coating with a PCR reaction mixture; and
(e) continuously expelling the PCR reaction mixture from the pipette tip, thereby generating a plurality of large uniform droplets.

15. The method of claim 14, wherein deforming the pipette tip orifice circumference comprises applying sufficient pressure between two surfaces to a longitudinal section of the pipette tip adjacent to and comprising the tip orifice for sufficient time to deform the orifice circumference to have an orifice aspect ratio of equal to or greater than about 3.5.

16. (canceled)

17. The method of claim 14, wherein the fluorosurfactant comprises RAN-008, Krytox, or a combination thereof at a concentration ranging from about 1 w/v % to about 5 w/v %, in the oil phase of HFE-7500 oil.

18. (canceled)

19. The method of claim 14, further comprising a water-soluble surfactant in an aqueous phase to stabilize the droplets in the oil phase during the ddPCR assay, wherein the water-soluble surfactant is polyoxyalkylene block copolymer surfactant Pluronic F127, Poloxamer 188, or a combination thereof, and is present in the aqueous phase at a concentration of ranging from about 0.01 w/v % to about 1.5 w/v %.

20-22. (canceled)

23. The method of claim 14, wherein the plurality of large uniform droplets comprises individual droplets having a size ranging from about 100 μm to about 400 μm, and comprises between about 1,000 to about 10,000 monodispersed droplets.

24-30. (canceled)

31. The method of claim 14, wherein the plurality of droplets is visually analyzed following the ddPCR assay using a camera or microscope.

32. (canceled)

33. The method of claim 14, wherein the pipette tip has an orifice aspect ratio ranging from about 3.5 to about 5.

34-41. (canceled)
